(19) United States
(12) Patent Application Publication
(10) Pub. No.: US 2025/0235479 A1
(43) Pub. Date: Jul. 24, 2025

(54) NATURAL KILLER CELLS EXPRESSING DUAL-TARGETING CHIMERIC ANTIGEN RECEPTORS FOR CD19 AND CD22, PRODUCTION METHOD AND USES THEREOF

(71) Applicants: INNOBATION BIO., LTD., Seoul (KR); UCI therapeutics Inc., Pohang-si (KR)

(72) Inventors: Su Yeong JEONG, Seoul (KR); Sang Won YOON, Seoul (KR); Jin Ho PARK, Seoul (KR); MinKoo SEO, Goyang-si (KR); Jun Ho LEE, Chuncheon-si (KR); Beom Jin KIM, Seoul (KR); Seung koo KIM, Yongin-si (KR); Ki Tae KIM, Seoul (KR)

(73) Assignees: INNOBATION BIO., LTD., Seoul (KR); UCI therapeutics Inc., Pohang-si (KR)

(21) Appl. No.: 19/074,276

(22) Filed: Mar. 7, 2025

Related U.S. Application Data
(63) Continuation-in-part of application No. 17/563,846, filed on Dec. 28, 2021, now Pat. No. 12,269,879.

(30) Foreign Application Priority Data
Dec. 30, 2020 (KR) 10-2020-0187510
Jun. 1, 2021 (KR) 10-2021-0070934

(51) Int. Cl. A61K 35/17; A61K 40/15; A61K 40/31; A61K 40/42; A61P 35/00; C07K 14/705; C07K 14/725; C07K 14/73; C07K 16/28; C12N 5/00; C12N 5/0783; C12N 15/86

(52) U.S. Cl. CPC A61K 35/17; A61K 40/15; A61K 40/31; A61K 40/4211; A61K 40/4212; A61P 35/00; C07K 14/7051; C07K 14/70514; C07K 14/70517; C07K 14/70521; C07K 14/70532; C07K 14/70596; C07K 16/2803; C12N 5/0018; C12N 5/0646; C12N 15/86; A61K 2239/13; A61K 2239/21; A61K 2239/22; A61K 2239/29; A61K 2239/31; C07K 2317/31; C07K 2317/565; C12N 2500/50; C12N 2501/02; C12N 2501/39; C12N 2502/00; C12N 2506/11; C12N 2740/15043

(57) ABSTRACT

The present disclosure relates to NK cells expressing dual-targeting chimeric antigen receptors for CD19 and CD22 and uses thereof, and more particularly, to a method for producing CAR-NK cells targeting CD19 and CD22, and a pharmaceutical composition for preventing or treating a disease mediated by B cells comprising a CAR-NK cell produced by the method. The present CAR-NK cells can be usefully utilized as a composition for preventing or treating diseases related to CD22 (or CD19) expression or diseases related to B cells.

### CROSS-REFERENCE TO RELATED APPLICATIONS

This application is a continuation-in-part of U.S. Non-Provisional application Ser. No. 17/563,846 filed on Dec. 27, 2021 which claims the benefit of Korean Patent Application No. 10-2020-0187510 filed Dec. 30, 2020 and Korean Patent Application No. 10-2021-0070934 filed on Jun. 1, 2021. The entire disclosures of those prior applications are hereby incorporated by reference.

### INCORPORATION BY REFERENCE OF SEQUENCE LISTING

The content of the electronically submitted sequence listing, file name: Q307077_sequence listing as filed.XLM; size 78,475 bytes; and date of creation of Mar. 5, 2025, filed herewith, is incorporated herein by reference in its entirety.

### TECHNICAL FIELD

The present invention relates to NK cells expressing dual-targeting chimeric antigen receptors for CD19 and CD22 and uses thereof, and more particularly, to a method for producing CAR-NK cells targeting CD19 and CD22, and a pharmaceutical composition for preventing or treating a disease mediated by B cells comprising a CAR-NK cell produced by the method.

### BACKGROUND ART

CD22 is expressed in most B cell leukemias and lymphomas, including NHL, acute lymphoblastic leukemia (B-ALL), chronic lymphocytic leukemia (B-CLL) and especially acute non-lymphocytic leukemia (ANLL).

Antibodies specific for CD22 have been developed for the treatment or diagnosis of diseases related to the CD22 expression, and the like. International Publication No. WO1998-041641 discloses a recombinant anti-CD22 antibody having a cysteine residue at the VH44 and V.100 positions, and International Publication No. WO 1998-042378 discloses an anti-CD22 antibody for the treatment of B-cell malignancies.

In order to produce antibodies for treatment as described above, monoclonal antibodies are usually produced using mice. However, non-human antibodies such as mouse-derived monoclonal antibodies are regarded as foreign antigens in the human body and thus have a problem in that the therapeutic effect is limited because they induce an immune response and have a short half-life.

In order to solve the above problems, a humanized antibody has been developed in which the remaining part except for only a region binding to an antigen of an antibody is substituted is a human antibody. As a currently used method for substituting a mouse antibody with a humanized antibody, a human antibody gene most similar to the antibody to be substituted is selected, and only a CDR region of the mouse antibody is substituted with a human antibody CDR position by a method called CDR grafting. The humanized antibody as described above has an advantage in that the immune response in the human body can be reduced because most of the genes are humanized.

Meanwhile, antibodies specific for the various other B cell surface markers (antigens) described above have been developed for the treatment of B cell diseases or disorders, autoimmune diseases, transplantation rejection responses, and the like, and in addition to the CD22 antigen, CD19 is an antigen that is commonly used as a target, and clinical studies on CAR-T cells targeting CD19 are also significantly progressing. However, there is a difference in the expression level of the target antigen depending on the cells such as leukemia that does not express CD19, and for a single CAR or single CAR-T cell therapy targeting only one antigen, a problem such as loss of the target antigen may occur due to the immune evasion strategy of tumor cells. In fact, in the case of patients with B cell acute lymphoblastic leukemia (ALL), CD19-negative recurrence in which CD19 was not expressed was observed (up to 25% of patients with B-cell ALL, who responded to CD19 CAR-T therapy for the first time), and this phenomenon was found to be a mechanism of tumor cell resistance to the CAR-T cell treatment (Maude, S. L., et al., N. Engl. I. Med., 378:439-448, 2018).

To solve this problem, CAR-T cells targeting double or multiple antigens have been studied, and targeting two antigens at the same time can reduce the possibility of antigen-loss variants.

### DISCLOSURE

### Technical Problem

In the present invention, a humanized anti-CD22 antibody was prepared by selecting an antibody binding to CD22 and using the antibody binding to CD22 in order to reduce an immune response in the human body, and using the humanized anti-CD22 antibody of the present invention, a chimeric antigen receptor and CAR-T cells were prepared.

Furthermore, a bispecific chimeric antigen receptor (bivalent CAR or bispecific CAR) targeting not only CD22 but also CD19 and bispecific CAR-T cells were prepared, and it was confirmed that the chimeric antigen receptor was normally expressed in the CD22-CAR-T cells and bispecific CD19xCD22-CAR-T cells prepared in the present invention, thereby completing the present invention.

An object of the present invention is to provide a humanized antibody specific for CD22, a polynucleotide encoding the antibody, a vector expressing the antibody, and a recombinant cell transformed with the vector.

Another object of the present invention is to provide a chimeric antigen receptor including the humanized antibody specific for CD22, a polynucleotide encoding the chimeric antigen receptor, a vector including the polynucleotide, and an immune effector cell expressing a chimeric antigen receptor, which includes the polynucleotide or vector.

Still another object of the present invention is to provide a bispecific chimeric antigen receptor including an antibody specific for CD19 and CD22, a polynucleotide encoding the bispecific chimeric antigen receptor, a vector including the polynucleotide, and an immune effector cell expressing a bispecific chimeric antigen receptor, and an immune effector cell expressing a bispecific chimeric antigen receptor, which includes the polynucleotide or vector.

Yet another object of the present invention is to provide a pharmaceutical composition including the immune effector cell for preventing or treating a disease mediated by B cells.

Another object of the present invention is to provide NK cell(s) expressing dual-targeting chimeric antigen receptors for CD19 and CD22, and more particularly, to NK cell(s) expressing the dual-targeting chimeric antigen receptors comprising a polynucleotide encoding the dual-targeting chimeric antigen receptor or a vector comprising the polynucleotide.

Another object of the present invention is to provide a method for producing CAR-NK cells targeting CD19 and CD22, and CAR-NK cells targeting CD19 and CD22 produced by the method.

Another object of the present invention is to provide a pharmaceutical composition for preventing or treating a disease mediated by B cells comprising CAR-NK cells targeting CD19 and CD22 prepared by the above methods.

Another object of the present invention is to provide a method for treating a disease mediated by B cells, comprising administering or injecting CAR-NK cells targeting CD19 and CD22 prepared by the above methods to an individual in need.

### Technical Solution

To achieve an object among the above-described objects,
the present invention provides a humanized antibody specifically binding to CD22 or a fragment thereof, including: a heavy chain variable region represented by an amino acid sequence of SEQ ID NO: 11 and a light chain variable region represented by an amino acid sequence of SEQ ID NO: 12; or
a heavy chain variable region represented by an amino acid sequence of SEQ ID NO: 15 and a light chain variable region represented by an amino acid sequence of SEQ ID NO: 16.

Further, the present invention provides a polynucleotide encoding the humanized antibody specifically binding to CD22 or the fragment thereof.

In addition, the present invention provides a vector including a polynucleotide encoding the humanized antibody specifically binding to CD22 or the fragment thereof.

Furthermore, the present invention provides a recombinant cell producing the humanized antibody specifically binding to CD22 transformed with the vector, or the fragment thereof.

To achieve another object,
the present invention provides a chimeric antigen receptor (CAR) targeting CD22, including: a CD22-binding domain; a transmembrane domain; a costimulatory domain; and an intracellular signal transduction domain,
in which the CD22-binding domain is a humanized antibody specifically binding to CD22 or a fragment thereof, including a heavy chain variable region represented by an amino acid sequence of SEQ ID NO: 11 and a light chain variable region represented by an amino acid sequence of SEQ ID NO: 12; or
a heavy chain variable region represented by an amino acid sequence of SEQ ID NO: 15 and a light chain variable region represented by an amino acid sequence of SEQ ID NO: 16.

In a preferred exemplary embodiment of the present invention, the transmembrane domain may be a protein selected from the group consisting of CD8α, CD4, CD28, CD137, CD80, CD86, CD152 and PD1, the costimulatory domain may be a protein selected from the group consisting of CD28, 4-1BB, OX-40 and ICOS, and the intracellular signal transduction domain may be derived from CD37.

In another preferred exemplary embodiment of the present invention, a hinge region located between the C-terminus of the CD22-binding domain and the N-terminus of a transmembrane domain may be further included, and the hinge region may be derived from CD8α.

Further, the present invention provides a polynucleotide encoding the chimeric antigen receptor.

In addition, the present invention provides a vector including a polynucleotide encoding the chimeric antigen receptor.

Furthermore, the present invention provides an immune effector cell including a polynucleotide encoding the chimeric antigen receptor or a vector including the polynucleotide, and expressing the chimeric antigen receptor.

To achieve another object,
the present invention provides a bispecific chimeric antigen receptor (CAR) targeting CD19 and CD22, including: a CD19-binding domain and a CD22-binding domain;
a transmembrane domain;
a costimulatory domain; and
an intracellular signal transduction domain,
in which the CD22-binding domain is an antibody specifically binding to CD22 or a fragment thereof, including a heavy chain variable region including a CDR1 region represented by an amino acid sequence of SEQ ID: 1, a CDR2 region represented by an amino acid sequence of SEQ ID NO: 2 and a CDR3 region represented by an amino acid sequence of SEQ ID NO: 3 and a light chain variable region including a CDR1 region represented by an amino acid sequence of SEQ ID NO: 4, a CDR2 region represented by an amino acid sequence of SEQ ID NO: 5 and a CDR3 region represented by an amino acid sequence of SEQ ID NO: 6.

In a preferred exemplary embodiment of the present invention, the CD19-binding domain and the CD22-binding domain may be linked in the order of a light chain variable region of an antibody specifically binding to CD19—a heavy chain variable region of an antibody specifically binding to CD22—a light chain variable region of an antibody specifically binding to CD22—a heavy chain variable region of an antibody specifically binding to CD19.

In another preferred exemplary embodiment of the present invention, the light chain variable region of the antibody specifically binding to CD19 may include a CDR1 region represented by an amino acid sequence of SEQ ID NO: 44, a CDR2 region represented by an amino acid sequence of SEQ ID NO: 45 and a CDR3 region represented by an amino acid sequence of SEQ ID NO: 46, and may be preferably represented by an amino acid sequence of SEQ ID NO: 48.

In still another exemplary embodiment of the present invention, the heavy chain variable region of the antibody specifically binding to CD19 may include a CDR1 region represented by an amino acid sequence of SEQ ID NO: 41, a CDR2 region represented by SEQ ID NO: 42 and a CDR3 region represented by an amino acid sequence of SEQ ID NO: 43, and may be preferably represented by an amino acid sequence of SEQ ID NO: 47.

In yet another preferred exemplary embodiment of the present invention, the transmembrane domain may be a protein selected from the group consisting of CD8α, CD4, CD28, CD137, CD80, CD86, CD152 and PD1, the costimulatory domain may be a protein selected from the group consisting of CD28, 4-1BB, OX-40 and ICOS, and the intracellular signal transduction domain may be derived from CD37.

In yet another preferred exemplary embodiment of the present invention, a hinge region located between the C-terminus of the CD22-binding domain and the N-terminus of a transmembrane domain may be further included, and the hinge region may be derived from CD8α.

Further, the present invention provides a polynucleotide encoding the bispecific chimeric antigen receptor.

In addition, the present invention provides a vector including a polynucleotide encoding the bispecific chimeric antigen receptor.

Furthermore, the present invention provides an immune effector cell including a polynucleotide encoding the bispecific chimeric antigen receptor or a vector including the polynucleotide, and expressing the bispecific chimeric antigen receptor.

To achieve another object,
the present invention provides a pharmaceutical composition including: the humanized antibody specifically binding to CD22 or the fragment thereof; or the immune effector cell for preventing or treating a disease mediated by B cells.

In a preferred exemplary embodiment of the present invention, the disease mediated by B cells may be selected from the group consisting of tumors, lymphoma, non-Hodgkin's lymphoma (NHL), aggressive NHL, relapsed aggressive NHL, relapsed indolent NHL, refractory NHL, refractory indolent NHL, chronic lymphocytic leukemia (CLL), small lymphocytic lymphoma (SLL), leukemia, hairy cell leukemia (HCL), acute lymphocytic leukemia (ALL), Burkitt's lymphoma and mantle cell lymphoma.

To achieve another object,
the present invention provides NK cells expressing the dual-targeting chimeric antigen receptors comprising a polynucleotide encoding the dual-targeting chimeric antigen receptor or a vector comprising the polynucleotide.

In addition, the present invention provides a method for producing CAR-NK cells targeting CD19 and CD22 comprising:
a step of treating peripheral blood mononuclear cells (PBMCs) expressing 70% or less of CD16, less than 10% of natural killer group 2D (NKG2D), 30% or less of CD57, 0.1% or more of low-density lipoprotein receptor (LDLR) and less than 10% of natural cytotoxicity triggering receptor 3 (NKp30) with dexamethasone to differentiate them into NK cells; and
a step of transducing the NK cells with a vector containing a polynucleotide encoding a bispecific chimeric antigen receptor (CAR) targeting CD19 and CD22;
wherein the bispecific chimeric antigen receptor (CAR) comprises a CD19-binding domain and a CD22-binding domain; a transmembrane domain; a costimulatory domain; and an intracellular signal transduction domain.

In a preferred exemplary embodiment of the present invention, the dexamethasone may be treated at the beginning (day 0) of induction of NK cell differentiation, and the induction of differentiation can be performed for 5 to 10 days in a medium containing dexamethasone.

In another preferred exemplary embodiment of the present invention, for inducing NK cell differentiation, the cells may be co-cultured with feeder cells expressing one or more proteins selected from the group consisting of IL-2, IL-15, IL-21, OX40L (CD134 ligand), and lunasin, and the feeder cells may be K562 cell lines, preferably irradiated K562 cell lines.

In another preferred exemplary embodiment of the present invention, the vector may be a viral vector, preferably a lentiviral vector, and the NK cells are treated with the viral vector at multiplicity of infection (MOI) of 3 to 7.

In another preferred exemplary embodiment of the present invention, prostaglandin E2 (PGE2) and polyoxyethylene-polyoxypropylene block copolymer may be additionally included to increase the expression rate of the chimeric antigen receptor (CAR) during the transduction process.

In another preferred exemplary embodiment of the present invention,
the heavy chain variable region of antibody specifically binding to CD22 may comprise a CDR1 region represented by an amino acid of SEQ ID: 1, a CDR2 region represented by an amino acid of SEQ ID NO: 2 and a CDR3 region represented by an amino acid of SEQ ID NO: 3;
the light chain variable region of antibody specifically binding to CD22 may comprise a CDR1 region represented by an amino acid of SEQ ID NO: 4, a CDR2 region represented by an amino acid of SEQ ID NO: 5 and a CDR3 region represented by an amino acid of SEQ ID NO: 6;
the heavy chain variable region of antibody specifically binding to CD19 may comprise a CDR1 region represented by an amino acid of SEQ ID: 41, a CDR2 region represented by an amino acid of SEQ ID NO: 42 and a CDR3 region represented by an amino acid of SEQ ID NO: 43; and
the light chain variable region of antibody specifically binding to CD19 may comprise a CDR1 region represented by an amino acid of SEQ ID NO: 44, a CDR2 region represented by an amino acid of SEQ ID NO: 45 and a CDR3 region represented by an amino acid of SEQ ID NO: 46.

In another preferred exemplary embodiment of the present invention, the present invention provides CAR-NK cells, which may be characterized by low expression of CD16, NKG2D, LDLR, and NKp30, and high expression of CD57.

In addition, the present invention provides a pharmaceutical composition for preventing or treating a disease mediated by B cells comprising CAR-NK cells targeting CD19 and CD22 prepared by the above methods.

In addition, the present invention provides a method for treating a disease mediated by B cells, comprising administering or injecting CAR-NK cells targeting CD19 and CD22 prepared by the above methods to an individual in need.

In another preferred exemplary embodiment of the present invention, the disease mediated by B cells can be selected from the group consisting of tumors, lymphoma, non-Hodgkin' s lymphoma (NHL), aggressive NHL, relapsed aggressive NHL, relapsed indolent NHL, refractory NHL, refractory indolent NHL, chronic lymphocytic leukemia (CLL), small lymphocytic lymphoma (SLL), leukemia, hairy cell leukemia (HCL), acute lymphocytic leukemia (ALL), Burkitt's lymphoma, and mantle cell lymphoma.

### Advantageous Effects

In the present invention, a humanized antibody specifically binding to CD22 was prepared, and single CAR-T cells targeting CD22 and bispecific CAR-T cells targeting CD19 and CD22 were prepared using the same.

It was confirmed that the CD22-CAR-T cells and bispecific CD19xCD22-CAR-T cells prepared in the present invention effectively recognized a CD22 antigen to activate CAR-T cells, and that the CD22-CAR-T cells and bispecific CD19xCD22-CAR-T cells effectively killed cells expressing CD22.

Furthermore, since it was confirmed that the bispecific CD19xCD22-CAR-T cells exhibited excellent antitumor effects in an animal model, the humanized antibody-based CD22-CAR-T cells and bispecific CD19xCD22-CAR-T cells specifically binding to CD22 of the present invention can be usefully utilized as a composition for preventing or treating a disease related to the expression of CD22 (or CD19) or a disease related to B cells.

Furthermore, it was confirmed that when CD16 is expressed in 70% or less, NKG2D (natural killer group 2D) is expressed in less than 10%, CD57 is expressed in less than 30%, LDLR (low-density lipoprotein receptor) is expressed in more than 0.1%, and NKp30 (natural cytotoxicity triggering receptor 3) is expressed in less than 10% among the cell surface proteins of PBMCs for producing CAR-NK cells (UCI-101), the delivery efficiency of the CAR expression vector and the efficiency of producing CAR-NK cells were surprisingly excellent.

In addition, the optimal conditions for inducing NK cell differentiation and the optimal conditions for transduction, which have excellent delivery efficiency of CAR expression vector and CAR-NK cell production efficiency, have been established, and the CAR-NK cells produced by the method of the present invention have been confirmed to exhibit excellent anti-tumor effects in animal models. Therefore, the present CAR-NK cells can be usefully utilized as a composition for preventing or treating diseases related to CD22 (or CD19) expression or diseases related to B cells.

### DESCRIPTION OF DRAWINGS

FIG. 1 illustrates the data confirming the binding force of the 2G1 antibody and the humanized 2G1 antibody selected in the present invention to CD22 by FACS.

FIG. 2 is a schematic view illustrating a chimeric antigen receptor (single CAR) targeting CD22.

FIG. 3 is a schematic view illustrating a method for preparing CD22-CAR-expressing cells using a lentivirus expressing CD22-CAR.

FIG. 4 is a schematic view illustrating (A) a method for transforming a HEK293 cell line with a CD22-CAR-expressing lentivirus and (B) a method for confirming the binding ability of the transformed HEK293 cells to a CD22 peptide.

FIG. 5 illustrates the data confirming the expression level of CD22-CAR in HEK293FT cells transformed with the lentivirus expressing 2G1(V4) and 2G1(V12)-based CD22-CAR, which are humanized anti-CD22 antibodies.

FIG. 6 a schematic view illustrating a method for preparing CD22-CART cells using a lentivirus expressing CD22-CAR.

FIG. 7 is a schematic view illustrating (A) a method for preparing CD22-CART cells using peripheral blood mononuclear cells (PBMCs) and (B) a method for confirming the binding ability of the prepared CD22-CAR-T cells to a CD22 peptide.

FIG. 8 illustrates the data confirming the CD22 binding ability of 2G1(V4) and 2G1(V12)-based CD22-CAR-T cells, which are humanized anti-CD22 antibodies.

FIG. 9 illustrates the data confirming the apoptotic effects on U2932 cells (CD22-expressing cells) and K562 cells (CD22-non-expressing cells) by 2G1(V4) and 2G1(V12)-based CD22-CAR-T cells, which are humanized anti-CD22 antibodies.

FIG. 10 illustrates the data confirming the apoptotic effects on NALM6 cells (CD22-expressing cells) and K562 cells (CD22-non-expressing cells) by 2G1(V4) and 2G1(V12)-based CD22-CAR-T cells, which are humanized anti-CD22 antibodies.

FIG. 11 is a schematic view illustrating a bispecific chimeric antigen receptor (bispecific CAR) targeting CD19 and CD22.

FIG. 12 is a schematic view illustrating a method for preparing CD19xCD22-CAR CD19-expressing cells using a lentivirus expressing bispecific CD19xCD22-CAR targeting CD19 and CD22.

FIG. 13 illustrates the data confirming the expression level of CD19xCD22 (V4)-CAR in HEK293FT cells transformed with a lentivirus expressing an anti-CD19 antibody FMC63 and humanized anti-CD22 antibody 2G1(V4)-based CD19xCD22-CAR.

FIG. 14 illustrates the data confirming the expression level of CD19xCD22 (V12)-CAR in HEK293FT cells transformed with a lentivirus expressing an anti-CD19 antibody and humanized anti-CD22 antibody 2G1(V12)-based CD19xCD22-CAR (V12).

FIG. 15 is a schematic view illustrating a method for preparing CD19xCD22-CAR-T cells using a lentivirus expressing CD19xCD22-CAR.

FIGS. 16a-16c illustrate the data confirming the activation of CD19xCD22-CAR-T cells. It was confirmed that in CD3+CD19xCD22-CAR-T cells (A), CD4+πCD19xCD22-CAR-T cells (B), and CD8+CD19xCD22-CAR-T cells (C), CD19xCD22-CAR-T cells bound to both a CD22 peptide and a CD19 peptide.

FIGS. 17a-17c illustrate the data confirming the expression level of IFNγ by (A) anti-CD19 antibody and mouse 2G1 antibody-based CD19xCD22 (mouse)-CAR-T cells, (B) anti-CD19 antibody and 2G1(V4) antibody-based CD19xCD22 (V4)-CAR-T cells, and (C) anti-CD19 antibody and 2G1(V12) antibody-based CD19xCD22 (V12)-CAR-T cells in the presence of target cells in order to confirm the activation of CD19xCD22-CAR-T cells.

FIGS. 18a and 18b illustrate the data confirming the apoptotic effects on (A) NALM6 cells (CD19 and CD22-expressing cells), (B) K562 cells (CD19 and CD22-non-expressing cells), K562/CD19+cells (CD19-expressing cells), K562/CD22+cells (CD22-expressing cells), and K562/CD19+/CD22+cells (CD19 and CD22-expressing cells) by CD19xCD22-CAR-T cells.

FIG. 19a illustrates images of luminescence expressed in NALM6/Luc cells injected into mice taken by IVIS Spectrum CT, and FIG. 19b illustrates data showing an antitumor effect by a viability curve of mice in experiments performed in FIG. 19a, in order to confirm the antitumor effects of CD19xCD22 (V4)-CAR-T cells.

FIG. 20 is a schematic diagram illustrating a method for producing CD19xCD22-CAR-NK cells using peripheral blood mononuclear cells (PBMCs).

FIG. 21 is a schematic diagram for establishing optimal treatment conditions for dexamethasone. In a of FIG. 21, dexamethasone was treated at a concentration of 100 nM or 1000 nM at the beginning of induction of NK cell differentiation (day 0), and then cultured under conditions of medium exchange after 1 hour of exposure, medium exchange after 24 hours of exposure, and exposure for 7 days without medium exchange. In addition, in b of FIG. 21, dexamethasone at a concentration of 100 nM or 1000 nM was treated 7 days after NK cell differentiation induction, and then cultured under conditions of medium exchange after 1 hour of exposure, medium exchange after 24 hours of exposure, and exposure for 7 days without medium exchange.

Additionally, in b of FIG. 21, dexamethasone at a concentration of 100 nM or 1000 nM was treated 7 days after the induction of NK cell differentiation and then incubated under the conditions of 1 hour exposure followed by medium change, 24 hours exposure followed by medium change, and 7 days exposure without medium change.

FIG. 22 shows data when dexamethasone was treated early in the induction of NK differentiation by the method of a of FIG. 21. a of FIG. 22 shows NK cell growth curves as a function of dexamethasone treatment concentration and exposure time, b of FIG. 22 shows PBMC population, and c of FIG. 22 shows NK cell expansion fold.

FIG. 23 shows data when dexamethasone was treated 7 days after induction of NK differentiation by the method of b of FIG. 21. a of FIG. 23 shows the NK cell growth curve as a function of dexamethasone treatment concentration and exposure time, b of FIG. 23 shows the PBMC growth, and c of FIG. 23 shows the NK cell expansion fold.

FIG. 24 shows the data of optimizing the transfection time to improve the CAR expression rate of CD19xCD22-CAR-NK cells. a of FIG. 24 is a schematic diagram of an experiment according to the transfection time of CD19xCD22-CAR lentiviral vector, and b of FIG. 24 is data confirming the CAR expression rate according to the transfection time of CD19xCD22-CAR lentiviral vector.

FIGS. 25a-25c show the data of optimizing the additives during transduction to improve the CAR expression rate of CD19xCD22-CAR-NK cells. FIG. 25a is data confirming the CAR expression rate according to the type of additive during transfection of CD19xCD22-CAR lentiviral vector, and FIG. 25b is data showing the CAR expression rate as a percentage. FIG. 25c is data showing CD19 and CD22 expression rates on the last day of culture (left), CD19 and CD22 expression rates as a function of culture day (center), and CD19xCD22-CAR-NK cell proliferation rate (right) of CD19xCD22-CAR-NK cells prepared by transduction under PGE2 +Lentiboost conditions.

FIG. 26 is data showing the optimized MOI of the CD19xCD22-CAR lentiviral vector to enhance the CAR expression rate of CD19xCD22-CAR-NK cells, and the data showing (a) CAR expression rate and (b) cell viability 7 days after transduction.

FIG. 27 is data showing (a) the growth rate of NK cells, (b) the growth rate of CD19xCD22-CAR-NK cells, and (c) CAR expression rate according to the donated PBMC type when the present final culture process established to enhance the CAR expression rate in NK cells was applied.

FIG. 28 is data showing the surface markers of CD19xCD22-CAR-NK cells according to the donated PBMC type when the present final culture process established to enhance the CAR expression rate on NK cells is applied.

FIG. 29 is data analyzing the marker expression patterns of donor-specific PBMCs to establish selection criteria for donated PBMCs.

FIG. 30 is data analyzing the phenotype of CD19xCD22-CAR-NK cells (UCI-101) produced by the optimal production method of the present invention. FIG. 30a shows the results of exogenous and endogenous CD37 western blotting analysis in periopheral blood NK cells (PBNKs) and UCI-101, and FIG. 30b shows data identifying markers of aging in PBNKs and UCI-101.

FIG. 31a shows the CAR expression rate and cytotoxicity against target cells (Raji cells and Daudi cells) of CD19xCD22-CAR-NK cells (UCI-101) produced by the optimal production method of the present invention, and FIG. 31b shows the degree of cytokine secretion when CD19xCD22-CAR-NK cells are co-cultured with target cells (Raji cells).

FIG. 32 is an image of the luminescence value of U2932-Luc cells when UCI-101 was administered once to the U2932 blood cancer animal model as shown in the schematic diagram in (a) to confirm the antitumor effect of CD19xCD22-CAR-NK cells (UCI-101), and (b) to evaluate the effect of UCI-101.

FIG. 33 shows the schematic diagram of (a) to confirm the antitumor effect according to the administration concentration of CD19xCD22-CAR-NK cells (UCI-101), when UCI-101 was repeatedly administered to the U2932 blood cancer animal model at low concentrations (2.5×10⁶ cells), medium concentrations (5×10⁶ cells), and high concentrations (1×10⁷ cells), (b) is data showing the image of the luminescence value of U2932-Luc cells to evaluate the efficacy of UCI-101.

FIG. 34 shows the schematic diagram of (a) to confirm the antitumor effect according to the administration concentration of CD19xCD22-CAR-NK cells (UCI-101), when UCI-101 was administered at a medium concentration (5×10⁶ cells) at 7-day intervals to the U2932 blood cancer animal model, and (b) is data that imaged the luminescence value of U2932-Luc cells to evaluate the efficacy of UCI-101.

FIG. 35 is data showing (a) CD19−/−, CD22−/−, and CD19−/−CD22−/−KO cell lines were generated to determine the antigen-specific cytotoxic activity of UCI-101, and CD19 and CD22 expression levels were determined, and (b) CD19 and CD22 expression levels of CD19xCD22-CAR-NK cells (UCI-101) and CAR-T cells were determined.

FIG. 36 is data showing (a) antigen-specific cytotoxicity and (b) cytokine secretion of CD19xCD22-CAR-NK cells and CD19xCD22-CAR-T cells when cocultured with CD19−/−, CD22−/−, and CD19−/−CD22−/−KO cell lines (U2932) prepared in FIG. 35, respectively.

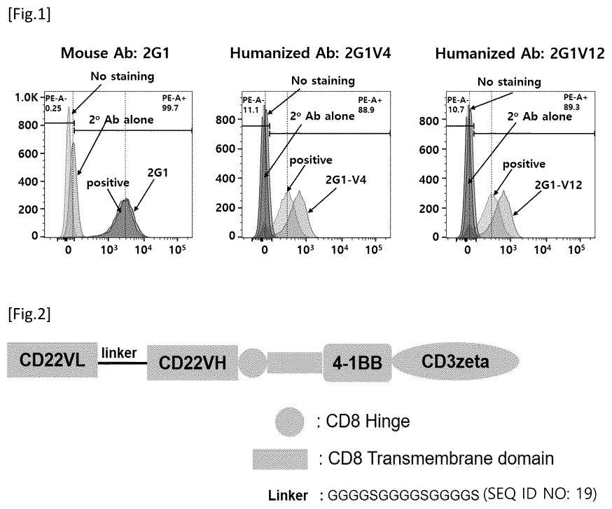

### MODES OF THE INVENTION

Hereinafter, the present invention will be described in detail.

### Humanized Antibody Specifically Binding to CD22

In an aspect, the present invention relates to a humanized antibody specifically binding to CD22 or a fragment thereof, including: a heavy chain variable region represented by an amino acid sequence of SEQ ID NO: 11 and a light chain variable region represented by an amino acid sequence of SEQ ID NO: 12; or
a heavy chain variable region represented by an amino acid sequence of SEQ ID NO: 15 and a light chain variable region represented by an amino acid sequence of SEQ ID NO: 16.

As used herein, the term “humanized antibody” refers to an antibody made using one of the techniques for possessing an amino acid sequence corresponding to an antibody produced by a human and/or making the human antibody as disclosed herein. Such a definition of the humanized antibody specifically excludes a humanized antibody including a non-human antigen binding residue.

In the present invention, the antibody may be a monoclonal antibody. As used herein, the term “monoclonal antibody” is also called a single clonal antibody, and is an antibody which is produced by single antibody-forming cells and is characterized as having a uniform primary structure (amino acid arrangement). The monoclonal antibody recognizes only one antigenic determinant and is generally produced by culturing a hybridoma cell, which is a fusion of cancer cells and antibody-producing cells, but may also be produced using other recombinant protein-expressing host cells using a secured antibody genetic sequence. In addition, the antibody may also be used by humanizing the remaining portion excluding the CDR portion, if necessary.

As used herein, the term “CDR”, that is, “complementarity determining region” refers to a non-contiguous antigen-binding site found in the variable regions of both heavy chain and light chain regions

As used herein, the term “antibody” may be used not only in a full form with two full-length light chains and two full-length heavy chains, but also in the form of a fragment of an antibody molecule. The fragment of an antibody molecule refers to a fragment at least having a peptide tag (epitope) binding function, and includes scFv, Fab, F(ab′), F(ab′), a single domain and the like.

Among the antibody fragments, Fab has one antigen-binding site with a structure having variable regions of a light chain and a heavy chain and a constant region of the light chain and a first constant region (CH1) of the heavy chain. Fab′ differs from Fab in that Fab′ has a hinge region including one or more cysteine residues at the C-terminus of the heavy chain CH1 domain. The F(ab′)2 antibody is produced while the cysteine residue of the hinge region of Fab′ forms a disulfide bond. Fv is a minimal antibody fragment having only a heavy chain variable region and a light chain variable region, a double chain Fv(dsFv) has a heavy chain variable region linked with a light chain variable region by a disulfide bond, and a single chain Fv(scFv) has a heavy chain variable region linked with a light chain variable region by a covalent bond generally through a peptide linker. Such an antibody fragment may be obtained using a proteolytic enzyme or preferably constructed by a gene recombination technique.

The monoclonal antibody specifically binding to CD22 of the present invention may be prepared using all or part of the CD22 protein as an immunogen (or an antigen). More specifically, first, immunization is achieved by subcutaneous, intramuscular, intravenous, intra-footpad or intraperitoneal injection of a CD22protein, a fusion protein including the CD22 protein, or a carrier containing the CD22protein as an immunogen into a mammal other than a human once to several times, together with an immune enhancer adjuvant (for example, a Freund's adjuvant) if necessary. The mammal other than the human is preferably a mouse, a rat, a hamster, a guinea pig, a chicken, a rabbit, a cat, a dog, a pig, a goat, a sheep, a donkey, a horse or a cow (including a transgenic animal manipulated to produce an antibody derived from other animals such as a transgenic mouse producing a human antibody), and more preferably a mouse, a rat, a hamster, a guinea pig, a chicken, or a rabbit. By performing immunization 1 to 4 times every 1 to 21 days from the initial immunization, antibody-producing cells can be obtained from the immune-sensitized mammal approximately 1 to 10 days after the final immunization. The number of immunizations and the time interval can be appropriately changed according to the characteristics and the like of the immunogen used.

Hybridomas secreting the monoclonal antibody can be prepared according to the method of Kohler and Milstein, et al. (Nature, 1975, Vol.256, p.495-497) or a method equivalent thereto. The hybridomas can be prepared by the cell fusion of antibody-producing cells included in any one selected from the group consisting of the spleen, lymph nodes, bone marrow or the tonsils, preferably the spleen, obtained from the animal other than a human, which is immune-sensitized as described above, with myeloma cells derived from a mammal lacking the ability to produce autoantibodies. The mammal may be a mouse, a rat, a guinea pig, a hamster, a chicken, a rabbit or a human, and may be preferably a mouse, a rat, a chicken, or a human.

For cell fusion, for example, a fusion accelerator such as polyethylene glycol or Sendai virus or a method using an electric pulse is used, and as an example, antibody-producing cells and mammal-derived cells capable of infinite proliferation are suspended in a fusion medium containing a fusion promoter at a ratio of about 1:1 to 1:10, and in this state, cultured at about 30 to 40° C. for about 1 to 5 minutes. As the fusion medium, for example, typical general media such as MEM medium, RPMI1640 medium, and Iscove's Modified Dulbecco's Medium may be used, and serum such as bovine serum is preferably excluded.

For a method for screening a hybridoma clone producing the monoclonal antibody, first, the fusion cells obtained as described above are transferred to a selection medium such as a HAT medium, and cultured at about 30 to 40° C. for about 3 days to 3 weeks to kill cells other than the hybridoma. Subsequently, the method may be performed by a method of culturing the hybridoma in a microtiter plate, and the like, and then searching for a portion where the reactivity between the immunogen used for an immune response of the animal other than the human as described above and a culture supernatant is increased by an immunoassay such as radioactive substance-marked immuno antibody (RIA) or Enzyme-Linked Immunosorbent Assay (ELISA). Also, a clone producing the monoclonal antibody found above shows a specific binding force to the immunogen.

The monoclonal antibody of the present invention may be obtained by culturing such a hybridoma in vitro or in vivo. For culturing, a typical method for culturing mammal-derived cells is used, and to collect a monoclonal antibody from a culture or the like, a typical method in this field for purifying antibodies in general is used. Examples of each method include salting out, dialysis, filtration, concentration, centrifugation, separatory sedimentation, ion-exchange chromatography, gel filtration chromatography, ion-exchange chromatography, affinity chromatography, high-performance liquid chromatography, gel electrophoresis, isoelectric point electrophoresis, and the like, and these are applied in combination, if necessary. The purified monoclonal antibody is then concentrated and dried to be made into a liquid or solid form depending on the application.

Further, the monoclonal antibody of the present invention may be obtained by synthesizing a gene which links DNA encoding variable regions of a heavy chain and a light chain to already-known DNA encoding each of the constant regions of the heavy chain and the light chain (for example, see Japanese Patent No. 2007-252372) by a PCR method or chemical synthesis, transplanting the gene into a known expression vector (pcDNA 3.1 (sold by Invitrogen)) that enables the expression of the gene, and the like to prepare a transformant, expressing the gene in a host such as CHO cells or E. coli to produce an antibody, and using Protein A or G columns and the like to purify the antibody from the culture solution.

In a specific exemplary embodiment of the present invention, in order to prepare an antibody specifically binding to CD22, an antibody (scFv) specifically binding to CD22 was selected by preparing and screening a hybridoma which produces an anti-CD22 antibody, and was named 2G1.

It was confirmed that the 2G1 antibody included a heavy chain variable region including a CDR1 region (GFSLTSYDI) represented by an amino acid sequence of SEQ ID NO: 1, a CDR2 region (IWTGGGT) represented by an amino acid sequence of SEQ

ID NO: 2 and a CDR3 region (VPHYYGYAMDYW) represented by an amino acid sequence of SEQ ID NO: 3 and a light chain variable region including a CDR1 region (QDINKY) represented by an amino acid sequence of SEQ ID NO: 4, a CDR2 region (YTS) represented by an amino acid sequence of SEQ ID NO: 5 and a CDR3 region (LQYDNLLT) represented by an amino acid sequence of SEQ ID NO: 6.

Specifically, it was confirmed that the 2G1 antibody included a heavy chain variable region represented by an amino acid sequence of SEQ ID NO: 7 and a light chain variable region represented by an amino acid sequence of SEQ ID NO: 8, the heavy chain variable region was encoded by a base sequence of SEQ ID NO: 9, and the light chain variable region was encoded by a base sequence of SEQ ID NO: 10.

In another specific exemplary embodiment of the present invention, a humanized antibody in which the anti-CD22 antibody 2G1 was modified to a structure corresponding to a human was prepared, and named 2G1(V4) and 2G1(V12).

The heavy chain variable region CDR and light chain variable region CDR of the 2G1(V4) and 2G1(V12) were similar to 2G1, and the remaining portion except for the CDR portion was humanized. Preferably, 2G1(V4) includes a heavy chain variable region represented by an amino acid sequence of SEQ ID: 11 and a light chain variable region represented by SEQ ID NO: 12, and the heavy chain variable region and light chain variable region of the 2G1(V4) antibody may be encoded by a base sequence of SEQ ID NO: 13 and a base sequence of SEQ ID NO: 14, respectively.

In addition, 2G1(V12) includes a heavy chain variable region represented by an amino acid sequence of SEQ ID NO: 15 and a light chain variable region represented by an amino acid sequence of SEQ ID NO: 16, and the heavy chain variable region and light chain variable region of the 2G1(V12) antibody may be encoded by a base sequence of SEQ ID NO: 17 and a base sequence of SEQ ID NO: 18, respectively.

The antibody specific for CD22 of the present invention is a single chain variable fragment (scFv), and may be constructed by a gene recombination technique such that the heavy chain variable region and the light chain variable region can be linked by a linker. The linker may be preferably represented by an amino acid sequence of SEQ ID NO: 19, or encoded by a base sequence of SEQ ID NO: 20, SEQ ID NO: 21, or SEQ ID NO: 22, but is not limited thereto.

When being linked as light chain variable region-linker-light chain variable region, the 2G1 antibody (mouse antibody) may be represented by an amino acid sequence of SEQ ID NO: 23, or encoded by a base sequence of SEQ ID NO: 24, the 2G1(V4) antibody may be represented by an amino acid sequence of SEQ ID NO: 25 or encoded by a base sequence of SEQ ID NO: 26, and the 2G1(V12) antibody may be represented by an amino acid sequence of SEQ ID NO: 27 or encoded by a base sequence of Seq ID NO: 28.

In another aspect, the present invention relates to a polynucleotide encoding the antibody specifically binding to CD22.

As used herein, the term “oligonucleotide” generally refers to a nucleic acid molecule, a deoxyribonucleotide or a ribonucleotide, or an analog thereof, separated by an arbitrary length. The polynucleotide may be prepared by (1) in-vitro amplification, such as polymerase chain reaction (PCR) amplification; (2) cloning and recombination; (3) purification such as digestion and gel electrophoresis separation; or (4) synthesis, such as chemical synthesis, and preferably, the separated polynucleotide may be prepared by a recombinant DNA technique. In the present invention, a polynucleotide for encoding an antibody or an antigen-binding fragment thereof may be prepared by various methods known in the art, including, but not limited to, the restriction fragment operation of synthetic oligonucleotides or the application of SOE PCR.

In still another aspect, the present invention relates to a vector including the polynucleotide encoding the antibody specifically binding to CD22, and a recombinant cell transformed with the vector.

As used herein, the term “expression vector” refers to a gene product including an essential regulatory element such as a promoter such that a target gene can be expressed in a suitable host cell. The vector may be selected from one or more of a plasmid, a retroviral vector and a lentiviral vector. Once transformed into a suitable host, the vector may be replicated and function independently of the host genome, or may be integrated into the genome itself in some cases.

Furthermore, the vector may include an expression control element that allows a coding region to be accurately expressed in a suitable host. Such a regulatory element is well known to those skilled in the art, and may include, for example, a promoter, a ribosome-binding site, an enhancer and other regulatory elements to regulate gene transcription or mRNA translation. A specific structure of an expression regulatory sequence may vary depending on the function of the species or cell type, but generally contains a 5′ non-transcriptional sequence and a 5′ or 3′ non-translational sequence that participate in transcription initiation and translation initiation, such as a TATA box, a capped cupping sequence, and a CAAT sequence, respectively. For example, a 5′ non-transcription expression regulatory sequence may include a promoter region capable of including a promoter sequence for transcribing and regulating functionally linked nucleic acids.

As used herein, the term “promoter” refers to a minimal sequence sufficient to direct transcription. Further, a promoter configuration sufficient to express a regulatory promoter-dependent gene induced by a cell type-specific or external signal or agent may be included, and the configurations may be located at the 5′ or 3′ portion of the gene. Both a conservative promoter and an inducible promoter are included. The promoter sequence may be derived from a prokaryote, eukaryote, or virus.

As used herein, the term “transformant” refers to a cell transformed by introducing a vector having a polynucleotide encoding one or more target proteins into a host cell, and examples of a method for preparing a transformant by introducing an expression vector into a host cell include a calcium phosphate method or calcium chloride/rubidium chloride method, electroporation, electroinjection, a chemical treatment method such as PEG, a method using a gene gun and the like, and the like described in the literature (Sambrook, J., et al., Molecular Cloning, A Laboratory Manual (2nd edition), Cold Spring Harbor Laboratory, 1.74, 1989).

A large amount of antibody protein can be prepared and separated by culturing a transformant in which the vector is expressed in a nutrient medium. Medium and culture conditions may be appropriately selected and used depending on the host cell. During culturing, conditions such as temperature, pH of the medium and duration of the culture should be adjusted appropriately so as to be suitable for growth of cells and mass production of proteins.

The vector according to the present invention may be transformed into a host cell, preferably a mammalian cell, for the production of antibodies. A number of suitable host cell lines capable of expressing a fully glycosylated protein have been developed in the art, and include COS-1 (for example, ATCC CRL 1650), COS-7 (for example, ATCC CRL-1651), HEK293, BHK21 (for example, ATCC CRL-10), CHO (for example, ATCC CRL 1610) and BSC-1 (for example, ATCC CRL-26) cell lines, Cos-7 cells, CHO cells, hep G2 cells, P3X63Ag8653, SP2/0-Agl4, 293 cells, Hela cells, and the like, and these cells are readily available, for example, from the ATCC (American Type Collection, USA). Preferred host cells include cells originating from lymphocytes such as melanoma and lymphoma cells.

### Chimeric Antigen Receptor Targeting CD22

In yet another aspect, the present invention relates to a chimeric antigen receptor (CAR) targeting CD22, including:
a CD22-binding domain;
a transmembrane domain;
a costimulatory domain; and
an intracellular signal transduction domain,
in which the CD22-binding domain is a humanized antibody specifically binding to CD22 or a fragment thereof, including a heavy chain variable region represented by an amino acid sequence of SEQ ID NO: 11 and a light chain variable region represented by an amino acid sequence of SEQ ID NO: 12; or
a heavy chain variable region represented by an amino acid sequence of SEQ ID NO: 15 and a light chain variable region represented by an amino acid sequence of SEQ ID NO: 16.

As used herein, the term “chimeric antigen receptor (CAR)” generally refers to a fusion protein containing an antigen and an extracellular domain having the ability to bind to one or more intracellular domains. CAR is a core part of the chimeric antigen receptor T cell (CAR-T), and may include an antigen binding domain, a transmembrane domain, costimulatory domain and an intracellular signal transduction domain. CAR may be combined with a T cell receptor-activated intracellular domain based on the antigen (for example, CD22) specificity of the antibody. CAR-expressing T cells with a modified gene may specifically identify and eliminate target antigen-expressing malignant cells.

As used herein, the term “CD22-binding domain” generally refers to a domain capable of specifically binding to a CD22 protein. For example, the CD22-binding domain may contain an anti-CD22 antibody or fragment thereof capable of specifically binding to a human CD22 polypeptide expressed in B cells or a fragment thereof.

As used herein, the term “binding domain” may be used interchangeably with “extracellular domain”, “extracellular binding domain”, “antigen-specific binding domain” and “extracellular antigen-specific binding domain”, and refers to a CAR domain or a fragment thereof which has the ability to specifically bind to a target antigen (for example, CD22).

In the present invention, the anti-CD22 antibody or the fragment thereof is a monoclonal antibody, preferably a single chain variable fragment (scFv) as the above-described anti-CD22 antibody. Specifically, the anti-CD22 antibody or the fragment thereof may be prepared using a 2G1(V4) or 2G1(V12) antibody, which is the humanized antibody specific to CD22 of the present invention.

In the present invention, the chimeric antigen receptor may be a bispecific chimeric antigen receptor further including a B cell surface marker-binding domain in addition to the CD22-binding domain, and the B cell surface marker may be CD1O, CD19, CD20, CD21, CD22, CD23, CD24, CD37, CD53, CD72, CD74, CD75, CD77, CD79a, CD79b, CD80, CD81, CD82, CD83, CD84, CD85 or CD86, and may be preferably CD19.

In the present invention, a signal peptide may be further included at the N-terminus of the CD22-binding domain, and the “signal peptide” generally refers to a peptide chain for guiding protein transduction. The signal peptide may be a short peptide having a length of 5 to 30 amino acids, and an amino acid sequence of SEQ ID NO: 36 was preferably used in the present invention.

In the present invention, a hinge region located between the C-terminus of the CD22-binding domain and the N-terminus of the transmembrane domain may be further included, and the hinge region is derived from CD8αand may be represented by an amino acid sequence of SEQ ID NO: 37. The “hinge region” generally refers to a linking region between an antigen-binding site and an immune cell Fc receptor (FcR)-binding region.

As used herein, the “transmembrane domain” generally refers to a domain of CAR that passes through a cell membrane and is linked to an intracellular signal transduction domain to play a role in signal transduction. The transmembrane domain may be derived from a protein selected from the group consisting of CD8α, CD4, CD28, CD137, CD80, CD86, CD152 and PD1, and may be preferably represented by an amino acid sequence of SEQ ID NO: 38.

As used herein, the “costimulatory domain” generally refers to an intracellular domain capable of providing an immunostimulatory molecule which is a cell surface molecule required for an effective response of lymphocytes to an antigen. The above-described costimulatory domain may include a costimulatory domain of CD28, may include a costimulatory domain of a TNF receptor family, such as a costimulatory domain of OX40 and 4-1BB, and may be preferably 4-1BB represented by an amino acid sequence of SEQ ID NO: 39.

As used herein, the “intracellular signal transduction domain” generally refers to a domain located inside a cell and capable of transducing a signal. In the present invention, the intracellular signal transduction domain is an intracellular signal transduction domain of a chimeric antigen receptor. For example, the intracellular signal transduction domain may be selected from an intracellular domain of CD37, an intracellular domain of CD28, an intracellular domain of CD28, an intracellular domain of 4-1BB and an intracellular domain of OX40, and may be preferably CD3Zrepresented by an amino acid sequence of SEQ ID NO: 40.

### Polynucleotide Encoding Chimeric Antigen Receptor and Chimeric Antigen Receptor Expression Vector

In yet another aspect, the present invention relates to a polynucleotide encoding the chimeric antigen receptor (CAR).

In the present invention, a polynucleotide encoding the chimeric antigen receptor (CAR) may include: a polynucleotide encoding a CD22-binding domain; a polynucleotide encoding a transmembrane domain; a polynucleotide encoding a costimulatory domain; and a polynucleotide encoding an intracellular signal transduction domain.

The polynucleotide encoding the CD22-binding domain may be a humanized 2G1(V4) or 2G1(V12) antibody specific for CD22 of the present invention, is in the form of a scFv in which a light chain variable region and a heavy chain variable region are linked by a linker, and a specific base sequence thereof is as described above.

Preferably, the polynucleotide encoding the chimeric antigen receptor (CAR) of the present invention may include: a 2G1(V4) antibody represented by a base sequence of SEQ ID NO: 26 or a 2G1(V12) antibody represented by a base sequence of SEQ ID NO: 28;
a transmembrane domain represented by a base sequence of SEQ ID NO: 32;
4-1BB (costimulatory domain) represented by a base sequence of SEQ ID NO: 33; and
CD3ζ (intracellular signal transduction domain) represented by a base sequence of SEQ ID NO: 34.

When a signal peptide is included at the N-terminus of the CD22-binding domain, a signal peptide represented by a base sequence of SEQ ID NO: 30 may further included. Further, a polynucleotide encoding a hinge region may be further included between the polynucleotide encoding the CD22-binding domain and the transmembrane domain, and may be preferably a CD8 hinge region represented by a base sequence of SEQ ID NO: 31.

In yet another aspect, the present invention relates to a vector including the polynucleotide encoding the chimeric antigen receptor (CAR).

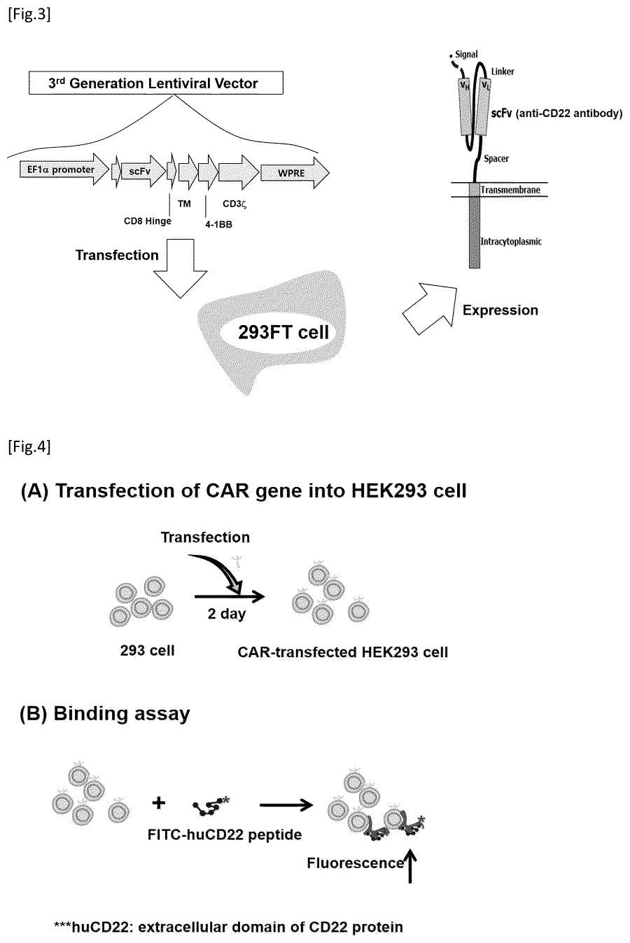

In the present invention, the vector is a recombinant viral vector, preferably, a lentiviral vector, and includes: an operably linked EF1α promoter; a polynucleotide encoding a signal peptide; a polynucleotide encoding a CD22-binding domain; a polynucleotide encoding a transmembrane domain; and a polynucleotide encoding an intracellular signal transduction domain, and may further include a woodchuck hepatitis virus post-transcriptional regulatory element (WPRE) in order to increase protein expression (FIG. 3).

The EF1α promoter may be represented by a base sequence of SEQ ID NO: 29, and may include a sequence which is 90% or more, 93% or more, 95% or more, 96% or more, 97% or more, 98% or more, or 99% or more identical to the base sequence of SEQ ID NO: 27, if necessary.

In addition, the promoter is operably linked to induce the expression of an anti-CD22 antibody (scFv), which is a CD22-binding domain.

In a specific exemplary embodiment of the present invention, as illustrated in FIG. 3, a lentiviral vector into which a polynucleotide encoding CD22-CAR was inserted was prepared, and CD22-CAR expressing cells were prepared by transforming 293 FT cells with the prepared vector. Furthermore, as illustrated in FIG. 5, it was confirmed that a chimeric antigen receptor targeting CD22 was expressed in the prepared CD22-CAR expressing cells.

A biological method for introducing a polynucleotide into a host cell includes the use of DNA and RNA vectors. Viral vectors and particularly retroviral vectors have become the most widely used methods for inserting genes into mammals, for example, human cells. Other viral vectors may be derived from lentiviruses, poxviruses, herpes simplex viruses, adenoviruses and adeno-related viruses, and the like.

A chemical means for introducing a polynucleotide into a host cell includes a colloidal dispersion system, such as a macromolecular complex, a nanocapsule, a microsphere, a bead, and a lipid-based system including an oil-in-water emulsion, a micelle, a mixed micelle, and a liposome. An exemplary colloidal system for use as a delivery vehicle in vitro and in vivo is a liposome (for example, an artificial membrane vesicle). Another method is available for a state-of-the-art delivery of nucleic acids, for example, a delivery of a polynucleotide using targeted nanoparticles or another suitable sub-micron-sized delivery system.

When a non-viral delivery system is used, an exemplary delivery vehicle is a liposome. The use of a lipid preparation is considered for the introduction of a nucleic acid into a host cell (in vitro, ex vivo or in vivo). In another aspect, the nucleic acid may be associated with a lipid. The nucleic acid associated with the lipid may be encapsulated in an aqueous solution of liposomes, interspersed in a bilayer of liposomes, attached to liposomes through a linking molecule associated with both liposomes and oligonucleotides, or captured in liposomes, or complexed with liposomes, or dispersed in a lipid-containing solution, or mixed with the lipid, or combined with the lipid, or contained in the lipid as a suspension, or contained or complexed with a micelle, or associated unlike the lipid. The lipid, lipid/DNA or lipid/expression vector association composition is not limited to any specific structure in a solution.

### Immune Effector Cell Expressing Chimeric Antigen Receptor (CAR)

In yet another aspect, the present invention relates to an immune effector cell, which includes a vector including a polynucleotide encoding the humanized antibody-based chimeric antigen receptor (CAR) specific for CD22 or a polynucleotide encoding the chimeric antigen receptor (CAR), and expresses the humanized antibody-based chimeric antigen receptor (CAR) specific for CD22.

In the present invention, the immune effector cell may be an isolated cell derived from mammals, preferably a T cell, a B cell, a natural killer (NK) cell, a dendritic cell, a bone marrow cell, a mononuclear cell or a macrophage, and more preferably a T cell.

In the present invention, the immune effector cell expressing the chimeric antigen receptor (CAR) may be prepared by introducing the CAR vector of the present invention into an immune effector cell, for example, a T cell or an NK cell.

Specifically, the CAR vector may be introduced into cells by methods known in the art, such as electroporation and Lipofectamine 2000 (Invitrogen). For example, the immune effector cell may be transfected with a lentiviral vector to integrate a viral genome carrying the CAR molecule into a host genome, ensuring long-term and stable expression of the target gene. As another example, a transposon may be used to introduce a CAR-transporting plasmid and a transferase-transporting plasmid into a target cell. As still another example, the CAR molecule may be added to the genome by a gene editing method (for example, CRISPR/Cas9).

An immune effector cell for preparing an immune effector cell expressing a chimeric antigen receptor (CAR) may be obtained from a subject, and the “subject” includes a living organism (for example, a mammal) in which an immune response can be elicited. Examples of the subject include a human, a dog, a cat, a mouse, a rat, and a transgenic species thereof. T cells may be obtained from numerous sources, including peripheral blood mononuclear cells, bone marrow, lymph node tissue, cord blood, thymic tissue, tissue from a site of infection, ascites, pleural effusion, spleen tissue, and tumors.

The T cells may be obtained from any number of techniques known to those skilled in the art, such as blood units collected from a subject using Ficoll™ separation. Cells from blood are obtained by apheresis or leukapheresis, and an apheresis product typically contains T cells, mononuclear cells, granulocytes, lymphocytes including B cells, other nucleated white blood cells, red blood cells, and platelets.

The cells collected by apheresis may be washed to remove a plasma fraction and to place the cells in an appropriate buffer or medium for subsequent processing steps. T cells are isolated from peripheral blood lymphocytes by lysing the red blood cells and depleting the monocytes, for example, by centrifugation through a PERCOLL™ gradient or by counterflow centrifugation.

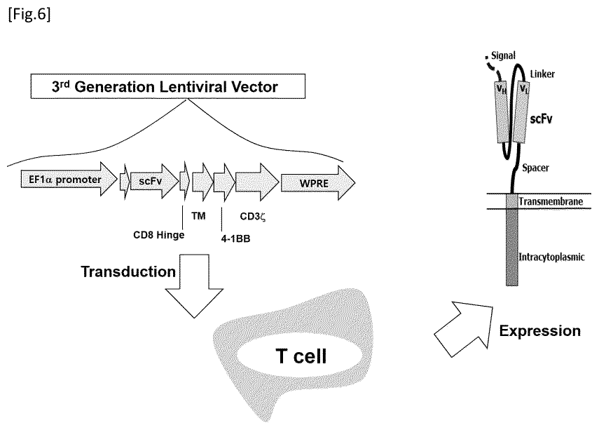

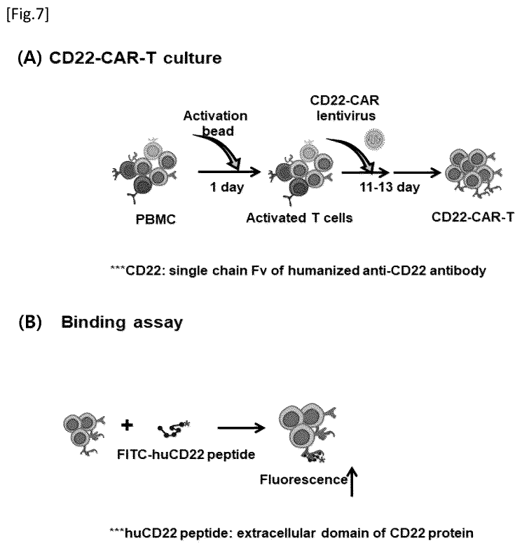

In still another specific exemplary embodiment of the present invention, as illustrated in FIGS. 6 and 7, activated T cells were isolated from peripheral blood monuclear cells (PBMCs), and then CD22-CAR-T cells were prepared by transducing a CD22-CAR lentivirus into T cells, and specifically, CD22-CAR-T cells were prepared using humanized 2G1(V4) and 2G1(V12), respectively.

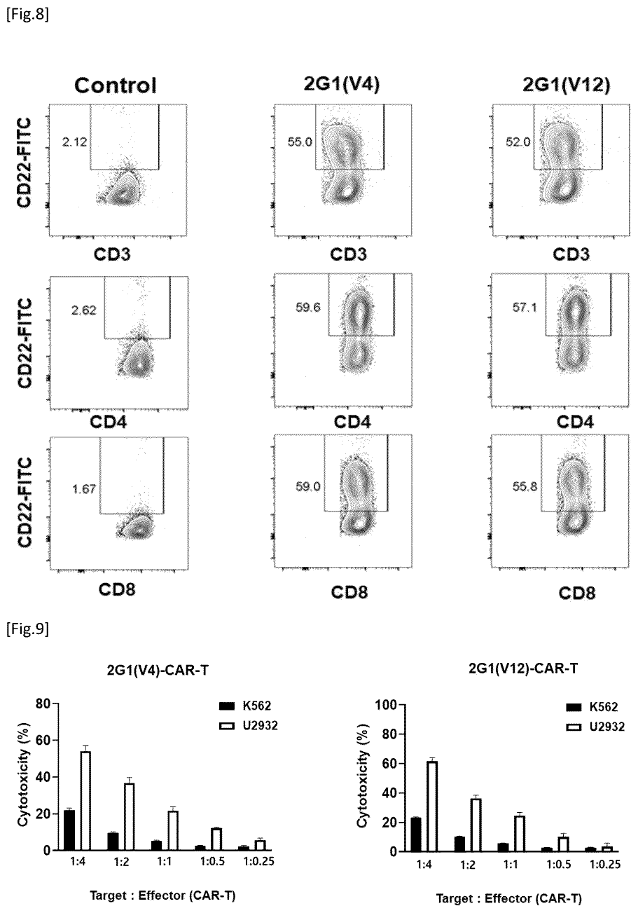

To confirm the activity of the prepared CD22-CAR-T cells, the CD22 peptide binding ability of CD22-CAR-T cells in which CD3, CD4 or CD8 was activated was confirmed. As illustrated in FIG. 8, it was confirmed that the CD22-CAR-T cells prepared in the present invention bound to the CD22 peptide.

In yet another specific exemplary embodiment of the present invention, as a result of confirming the apoptotic effect on the target cell by the CD22-CAR-T cells, as illustrated in FIG. 9, it was confirmed that the CD22-CAR-T cells specifically exhibited an apoptotic effect on U2932 cells and NALM6 cells expressing CD22.

That is, the humanized anti-CD22 antibody (2G1(V4) and 2G1(V12))-based chimeric antigen receptor of the present invention and CAR-T cells using the same may be usefully used as a composition for preventing or treating a disease related to

B cells or CD22 expression.

### Bispecific Chimeric Antigen Receptor (CAR) Targeting CD19/CD22

In yet another aspect, the present invention relates to a bispecific antigen receptor targeting CD19 and CD22 (CD19xCD22 bispecific CAR), including:
a CD19-binding domain and a CD22-binding domain;
a transmembrane domain;
a costimulatory domain; and
an intracellular signal transduction domain,
in which the CD22-binding domain is an antibody specifically binding to CD22 or a fragment thereof, including a heavy chain variable region including a CDR1 region represented by an amino acid sequence of SEQ ID: 1, a CDR2 region represented by an amino acid sequence of SEQ ID NO: 2 and a CDR3 region represented by an amino acid sequence of SEQ ID NO: 3 and a light chain variable region including a CDR1 region represented by an amino acid sequence of SEQ ID NO: 4, a CDR2 region represented by an amino acid sequence of SEQ ID NO: 5 and a CDR3 region represented by an amino acid sequence of SEQ ID NO: 6.

In the present invention, the antibody specifically binding to CD22 or the fragment thereof is an anti-CD22 antibody including a heavy chain variable region represented by an amino acid sequence of SEQ ID NO: 7 and a light chain variable region represented by an amino acid sequence of SEQ ID NO: 8, or
may be a humanized anti-CD22 antibody including: a heavy chain variable region represented by an amino acid sequence of SEQ ID NO: 11 and a light chain variable region represented by an amino acid sequence of SEQ ID NO: 12; or
a heavy chain variable region represented by an amino acid sequence of SEQ ID NO: 15 and a light chain variable region represented by an amino acid sequence of SEQ ID NO: 16.

In the present invention, the bispecific or bivalent chimeric antigen receptor is a CAR capable of simultaneously binding to two different types of antigens, and in the present invention, preferably, a bispecific chimeric antigen receptor targeting both CD19 and CD22 was prepared, and in the CD19-binding domain, any known anti-CD19 antibody sequence can be used without limitation.

A specific content on the chimeric antigen receptor is as described above, and the CD19-binding domain and CD22-binding domain of the bispecific chimeric antigen receptor were linked in the form of a loop (LoopCAR) as illustrated in FIG. 11. That is, the CD19-binding domain and the CD22-binding domain may be linked in the order of a light chain variable region (CD19VL) of an antibody specifically binding to CD19—a heavy chain variable region (CD22VH) of an antibody specifically binding to CD22—a light chain variable region (CD22VL) of an antibody specifically binding to CD22—a heavy chain variable region (CD19VH) of an antibody specifically binding to CD19.

The light chain variable region of the antibody specifically binding to CD19 may include a CDR1 region represented by an amino acid sequence of SEQ ID NO: 44, a CDR2 region represented by an amino acid sequence of SEQ ID NO: 45 and a CDR3 region represented by an amino acid sequence of SEQ ID NO: 46, and may be preferably represented by an amino acid sequence of SEQ ID NO: 48.

The heavy chain variable region of the antibody specifically binding to CD19 may include a CDR1 region represented by an amino acid sequence of SEQ ID NO: 41, a CDR2 region represented by SEQ ID NO: 42 and a CDR3 region represented by an amino acid sequence of SEQ ID NO: 43, and may be preferably represented by an amino acid sequence of SEQ ID NO: 47.

The CD19-binding domain and the CD22-binding domain may be manufactured by a gene recombination technique so as to be linked by a linker, CD19VL and CD22VH or CD22VL and CD19VH may be preferably linked by a linker (linker 1 in FIG. 11) represented by an amino acid sequence of SEQ ID NO: 51, CD22VH and CD22VL may be linked by a linker (linker 6 in FIG. 11) represented by an amino acid sequence of SEQ ID NO: 54, but the present invention is not limited thereto, and a peptide including any amino acid sequence that does not affect the antibody activity can be used.

### Polynucleotide Encoding Bispecific Chimeric Antigen Receptor Targeting CD19/CD22 and Bispecific Chimeric Antigen Receptor Expression Vector Targeting CD19/CD22

In yet another aspect, the present invention relates to a polynucleotide encoding the bispecific chimeric antigen receptor targeting CD19 and CD22.

In the present invention, the polynucleotide encoding the bispecific chimeric antigen receptor may include: a polynucleotide encoding a CD19-binding domain and a polynucleotide encoding a CD22-binding domain; a polynucleotide encoding a transmembrane domain; a polynucleotide encoding a costimulatory domain; and a polynucleotide encoding an intracellular signal transduction domain.

Preferably, the polynucleotide encoding the bispecific chimeric antigen receptor of the present invention may include:
a bispecific antibody including a light chain variable region (CD19VL; SEQ ID NO: 50) of an antibody specifically binding to CD19—a heavy chain variable region (CD22VH; SEQ ID NO: 9, SEQ ID NO: 13 or SEQ ID NO: 17) of an antibody specifically binding to CD22—a light chain variable region (CD22VL; SEQ ID NO: 10, SEQ ID NO: 14 or SEQ ID NO: 18) of an antibody specifically binding to CD22—a heavy chain variable region (CD19VH; SEQ ID NO: 49) of an antibody specifically binding to CD19;
a transmembrane domain represented by a base sequence of SEQ ID NO: 32;
4-1BB (costimulatory domain) represented by a base sequence of SEQ ID NO: 33; and
CD3ζ (intracellular signal transduction domain) represented by a base sequence of SEQ ID NO: 34.

When the ‘CD19VL and CD22VH’ or ‘CD22VL and CD19VH’ are linked by a linker represented by an amino acid sequence of SEQ ID NO: 51, a polynucleotide for the linker may be represented by a base sequence of SEQ ID NO: 52 or SEQ ID NO: 53, and when the ‘CD22VH and CD22VL’ are linked by a linker represented by an amino acid sequence of SEQ ID NO: 54, a polynucleotide for the linker may be represented by a base sequence of SEQ ID NO: 55, but the present invention is not limited thereto, and a polynucleotide encoding any amino acid sequence that does not affect the antibody activity can be used.

When a signal peptide is included at the N-terminus of the CD19/CD22-binding domain, a signal peptide represented by a base sequence of SEQ ID NO: 30 may further included. Further, a polynucleotide encoding a hinge region may be further included between the polynucleotide encoding the CD22-binding domain and the transmembrane domain, and may be preferably a CD8 hinge region represented by a base sequence of SEQ ID NO: 31.

In yet another aspect, the present invention relates to a vector including the polynucleotide encoding the bispecific chimeric antigen receptor.

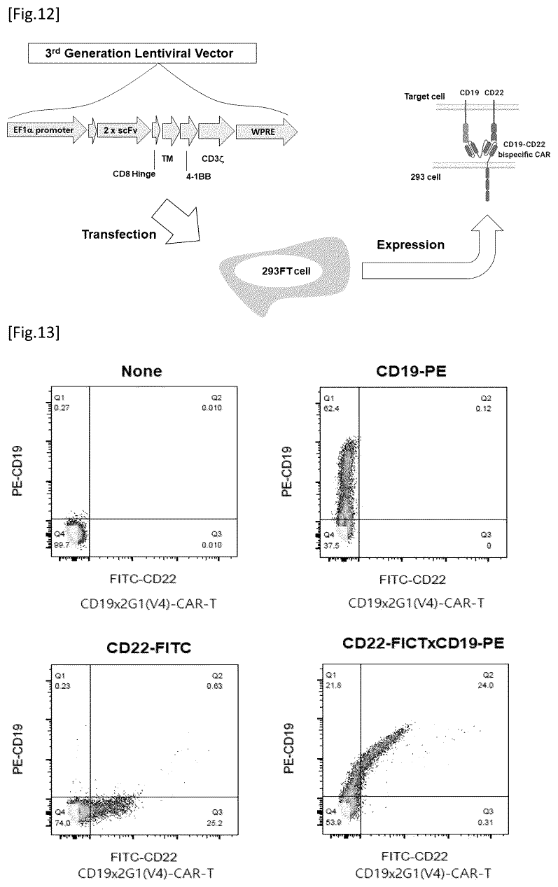

In the present invention, the vector is a recombinant viral vector, preferably, a lentiviral vector, and includes: an operably linked EF1a promoter; a polynucleotide encoding a signal peptide; a polynucleotide encoding a CD19-binding domain and a CD22-binding domain; a polynucleotide encoding a transmembrane domain; and a polynucleotide encoding an intracellular signal transduction domain, and may further include a woodchuck hepatitis virus post-transcriptional regulatory element (WPRE) in order to increase protein expression (FIG. 12).

The EF1α promoter may be represented by a base sequence of SEQ ID NO: 29, and may include a sequence which is 90% or more, 93% or more, 95% or more, 96% or more, 97% or more, 98% or more, or 99% or more identical to the base sequence of SEQ ID NO: 27, if necessary.

In addition, the promoter is operably linked to induce the expression of the anti-CD19/CD22 antibody (CD19VL-CD22VH-CD22VL-CD19VH), which is a CD19xCD22-binding domain, and a specific content on the vector is described above.

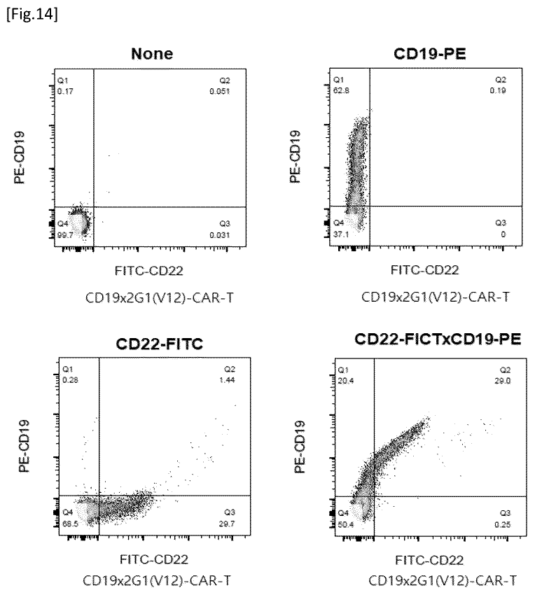

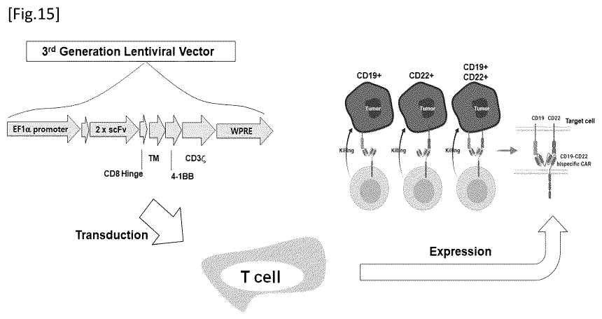

In a specific exemplary embodiment of the present invention, as illustrated in FIG. 12, a lentiviral vector into which a polynucleotide encoding CD19xCD22-CAR was inserted was prepared, and CD19xCD22-CAR expressing cells were prepared by transforming 293 FT cells with the prepared vector. Furthermore, as illustrated in FIGS. 13 and 14, it was confirmed that a bispecific antigen receptor targeting CD19 and CD22 was expressed in the prepared CD19xCD22-CAR expressing cells.

### Immune Effector Cell Expressing Bispecific Chimeric Antigen Receptor Targeting CD19xCD22

In yet another aspect, the present invention relates to an immune effector cell, which includes a polynucleotide encoding the bispecific chimeric antigen receptor (CAR) or a vector including the polynucleotide encoding the bispecific chimeric antigen receptor (CAR), and expresses the bispecific chimeric antigen receptor (CAR).

In the present invention, the immune effector cell may be an isolated cell derived from mammals, preferably a T cell, a B cell, a natural killer (NK) cell, a dendritic cell, a bone marrow cell, a mononuclear cell or a macrophage, and more preferably a T cell. Further, a specific content on the chimeric antigen receptor-expressing immune effector cell is as described above.

In a specific exemplary embodiment of the present invention, activated T cells were isolated from peripheral blood mononuclear cells (PBMCs) by the same method as for the preparation of CD22-CAR-T cells (FIG. 7), and then CD19xCD22-CAR-T cells were prepared by transducing a CD19xCD22-CAR lentivirus into T cells, and specifically, CD19xCD22-CAR-T cells were prepared using a 2G1 antibody (mouse), and humanized 2G1(V4) and 2G1(V12), respectively.

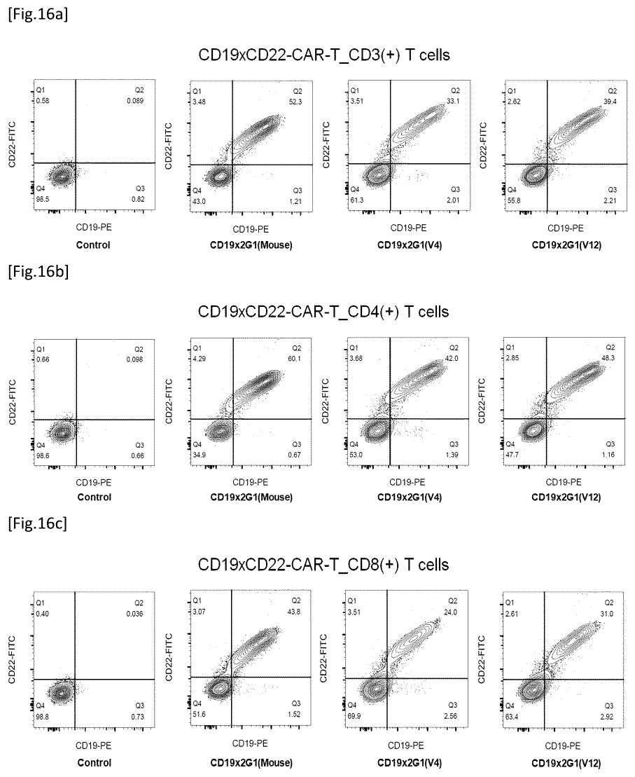

To confirm the activity of the prepared CD19xCD22-CAR-T cells, the CD22 peptide and CD19 peptide binding ability of CD19xCD22-CAR-T cells in which CD3, CD4 or CD8 was activated was confirmed. As illustrated in FIGS. 16a-16c, it was confirmed that the CD19xCD22-CAR-T cells prepared in the present invention bound to both the CD22 peptide and the CD19 peptide.

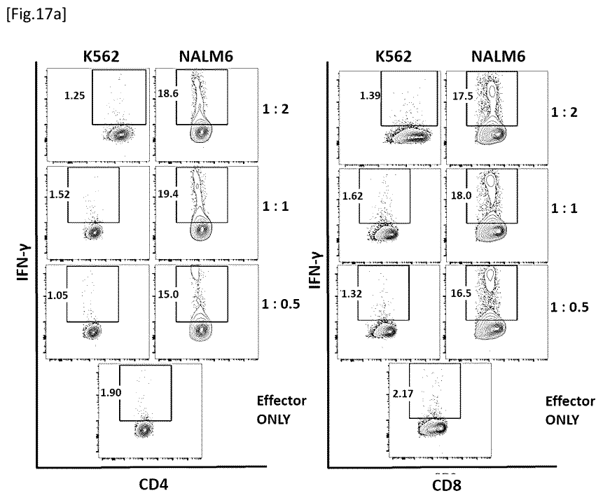

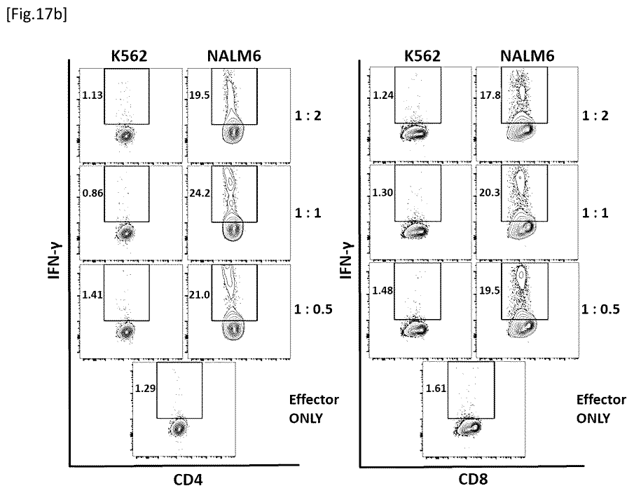

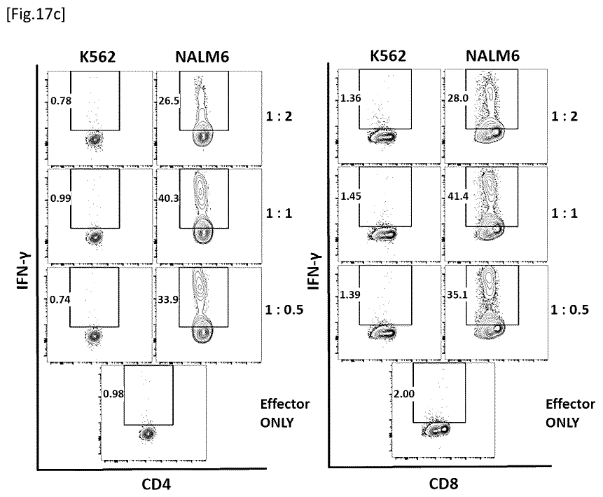

In another specific exemplary embodiment of the present invention, to confirm the activation of CD19xCD22-CAR-T cells, the expression level of IFNγ by CD19xCD22-CAR-T cells was confirmed in the presence of a target cell. As a result, as illustrated in FIGS. 17a to 17c, it was confirmed that T cells were not activated in K562 cells that do not express CD19 and CD22, whereas T cells were activated in the presence of NALM6 cells expressing CD19 and CD22, thereby increasing the expression of IFNγ.

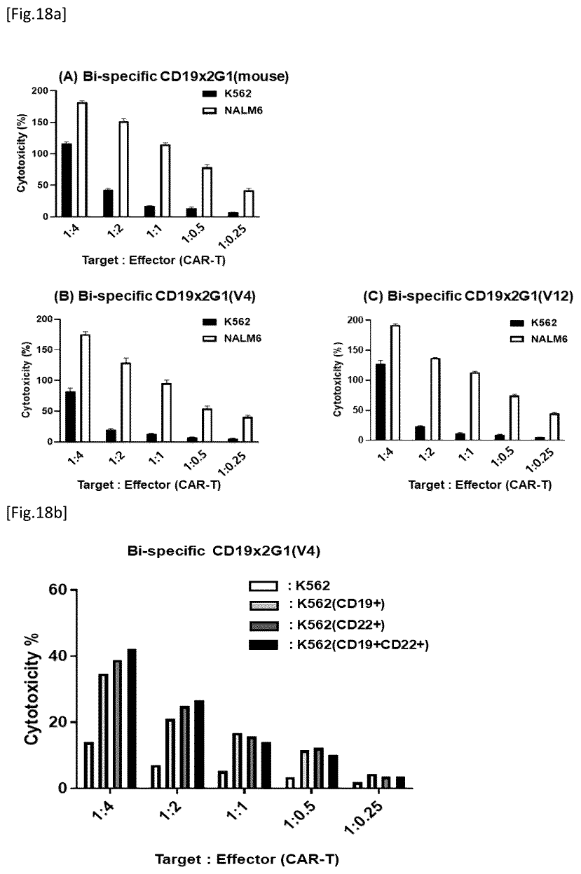

In another specific exemplary embodiment of the present invention, as a result of confirming the apoptotic effect on the target cell by the CD19xCD22-CAR-T cells, as illustrated in FIGS. 18a and 18b, it was confirmed that the CD19xCD22-CAR-T cells specifically exhibited an apoptotic effect on NALM6 cells expressing CD19 and CD22.

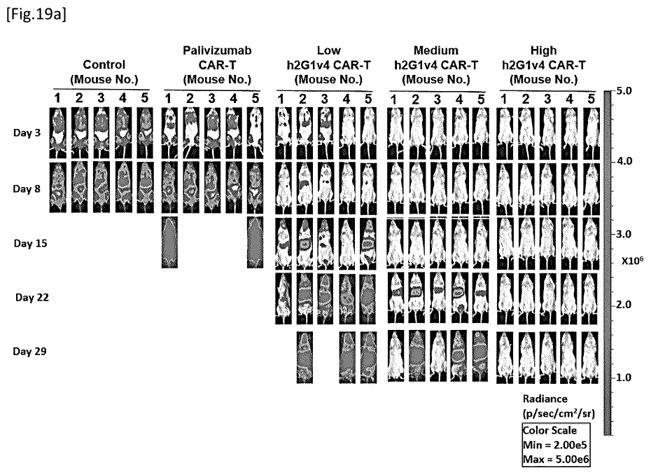

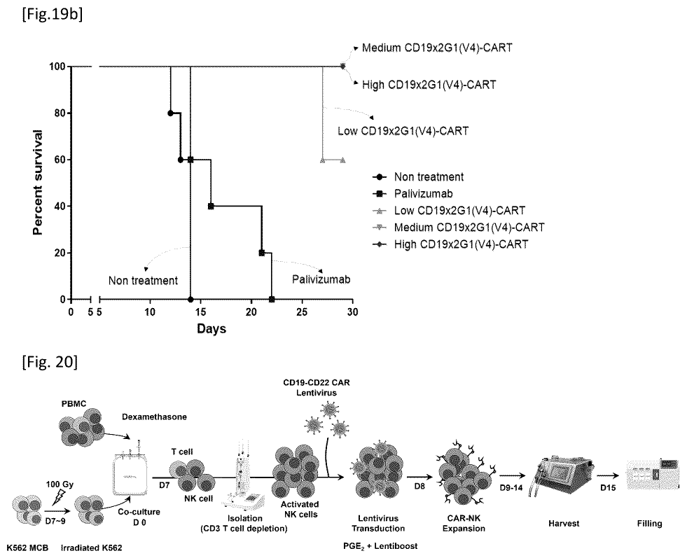

Furthermore, in still another specific exemplary embodiment of the present invention, to confirm the antitumor effects of CD19xCD22-CAR-T cells, CD19xCD22-CAR-T cells prepared using a humanized 2G1-V4 antibody were injected intratumorally into a mouse model xenografted with tumor cells, and then changes in tumor size were observed. As a result, as illustrated in FIGS. 19a and 19b, it was confirmed that the antitumor effects of the CD19xCD22-CAR-T cells of the present invention were excellent compared to those of antibody-based palivizumab-CAR-T cells binding to the SV40 virus used as a positive control, and it was confirmed that when CD19xCD22-CAR-T cells were treated at 5x106 or more, most of the tumor cells were killed, and thus no tumor tissue was observed.

That is, the bispecific chimeric antigen receptor targeting the CD19xCD22 of the present invention and CD19xCD22-CAR-T cells may be usefully used as a composition for preventing or treating a disease related to B cells or CD19xCD22 expression.

### NK Cell Expressing A Bispecific Chimeric Antigen Receptor Targeting CD19xCD22 and a Method for Producing the Same

In yet another aspect, the present invention relates to NK cell(s) expressing the dual-targeting chimeric antigen receptors comprising a polynucleotide encoding the dual-targeting chimeric antigen receptor or a vector comprising the polynucleotide encoding the dual-targeting chimeric antigen receptor.

In addition, the present invention relates to a method for producing CAR-NK cells targeting CD19 and CD22 comprising: a step of treating peripheral blood mononuclear cells (PBMCs) expressing 70% or less of CD16, less than 10% of natural killer group 2D (NKG2D), 30% or less of CD57, 0.1% or more of low-density lipoprotein receptor (LDLR) and less than 10% of natural cytotoxicity triggering receptor 3 (NKp30) with dexamethasone to differentiate them into NK cells; and
a step of transducing the NK cells with a vector containing a polynucleotide encoding a bispecific chimeric antigen receptor (CAR) targeting CD19 and CD22;
wherein the bispecific chimeric antigen receptor (CAR) comprises a CD19-binding domain and a CD22-binding domain; a transmembrane domain; a costimulatory domain; and an intracellular signal transduction domain.

In addition, the present invention more specifically relates to a method for producing CAR-NK cells targeting CD19 and CD22 comprising:
(a) a step of treating peripheral blood mononuclear cells (PBMCs) separated from blood with dexamethasone to induce differentiation into NK cells and isolating activated NK cells; and
(b) a step of transducing the activated NK cells with a vector comprising a polynucleotide encoding a bispecific chimeric antigen receptor (CAR) targeting CD19 and CD22;
wherein the PBMC of step (a) expresses CD16 at 70% or less, NKG2D (natural killer group 2D) at less than 10%, CD57 at 30% or less, LDLR (low-density lipoprotein receptor) at 0.1% or more, and NKp30 (natural cytotoxicity triggering receptor 3) at less than 10%; and
the bispecific chimeric antigen receptor (CAR) comprises a CD19-binding domain and a CD22-binding domain; a transmembrane domain; a costimulatory domain; and an intracellular signal transduction domain.

In the present invention, the dexamethasone can be treated at the beginning (day 0) of induction of NK cell differentiation, and the induction of differentiation can be performed for 5 to 10 days in a medium containing dexamethasone.

In the present invention, the NK cell may be co-cultured with a feeder cell expressing OX40L (CD134 ligand) for induction of NK cell differentiation, and the feeder cell may be K562 cell lines, preferably irradiated K562 cell lines.

In the present invention, the vector may be a viral vector, preferably a lentiviral vector, and the NK cells are treated with the viral vector at multiplicity of infection (MOI) of 3 to 7.

In the present invention, the method may further comprise prostaglandin E2 (PGE2) and poloxamer synperonic F108 (Non-ionic, Amphiphilic poloxamer synperonic F108, LentiBOOST) to increase the expression rate of the chimeric antigen receptor (CAR) during the step of transducing.

In a specific embodiment of the present invention, as a result of establishing the optimal conditions for improving the CAR expression rate of CD19xCD22-CAR-NK cells, (1) dexamethasone was treated at the beginning (day 0) of induction of NK differentiation, differentiation was induced for 5 to 10 days (preferably 7 days) in a medium containing dexamethasone, (2) NK cells induced for differentiation in (1) were treated at 3 to 7 MOI (preferably 5 MOI) when transducing CD19xCD22-CAR lentiviral vector, and (3) a culture process was established that additionally included PGE2 and LentiBOOST to increase the expression rate of chimeric antigen receptor (CAR) during transduction.

In addition, when CD16 was expressed at 70% or less, NKG2D (natural killer group 2D) was less than 10%, CD57 was expressed at 30% or less, LDLR (low-density lipoprotein receptor) was 0.1% or more, and NKp30 (Natural cytotoxicity triggering receptor 3) was expressed at 10% or less among the cell surface proteins of PBMC for producing CAR-NK cells (UCI-101), it was confirmed that the delivery efficiency of the CAR expression vector and the CAR-NK cell production efficiency were excellent, and it was confirmed that the CD19xCD22-CAR-NK cells produced by the method of the present invention exhibited excellent anti-tumor effects in animal models.

A specific embodiment on the chimeric antigen receptor is as described above, and the CD19-binding domain and CD22-binding domain of the bispecific chimeric antigen receptor may be linked in the form of a loop (LoopCAR) as illustrated in FIG. 11. That is, the CD19-binding domain and the CD22-binding domain may be linked in the order of a light chain variable region (CD19VL) of an antibody specifically binding to CD19—a heavy chain variable region (CD22VH) of an antibody specifically binding to CD22—a light chain variable region (CD22VL) of an antibody specifically binding to CD22—a heavy chain variable region (CD19VH) of an antibody specifically binding to CD19.

The light chain variable region of the antibody specifically binding to CD19 may include a CDR1 region represented by an amino acid sequence of SEQ ID NO: 44, a CDR2 region represented by an amino acid sequence of SEQ ID NO: 45 and a CDR3 region represented by an amino acid sequence of SEQ ID NO: 46, and may be preferably represented by an amino acid sequence of SEQ ID NO: 48.

The heavy chain variable region of the antibody specifically binding to CD19 may include a CDR1 region represented by an amino acid sequence of SEQ ID NO: 41, a CDR2 region represented by SEQ ID NO: 42 and a CDR3 region represented by an amino acid sequence of SEQ ID NO: 43, and may be preferably represented by an amino acid sequence of SEQ ID NO: 47.

The CD19-binding domain and the CD22-binding domain may be manufactured by a gene recombination technique so as to be linked by a linker, CD19VL and CD22VH or CD22VL and CD19VH may be preferably linked by a linker (linker 1 in FIG. 11) represented by an amino acid sequence of SEQ ID NO: 51, CD22VH and CD22VL may be linked by a linker (linker 6 in FIG. 11) represented by an amino acid sequence of SEQ ID NO: 54, but the present invention is not limited thereto, and a peptide including any amino acid sequence that does not affect the antibody activity can be used.

### Composition for Preventing or Treating Disease Mediated by B Cells or Disease Mediated by CD19xCD22 Expression

In yet another aspect, the present invention relates to a pharmaceutical composition for preventing or treating a disease mediated by B cells, including: a humanized antibody specifically binding to CD22; an immune effector cell expressing a chimeric antigen receptor targeting CD22; or an immune effector cell expressing a bispecific chimeric antigen receptor specifically binding to CD19xCD22.

In yet another aspect, the present invention relates to a pharmaceutical composition for preventing or treating a disease mediated by B cells comprising CAR-NK cells targeting CD19 and CD22 prepared by the above methods.

In addition, the present invention relates to a method for treating a disease mediated by B cells, comprising administering or injecting CAR-NK cells targeting CD19 and CD22 prepared by the above methods to an individual in need.

Preferably, the NK cell(s) can be manufactured by the CD19xCD22-CAR-NK cell production method described above, and the CAR-NK cells may be characterized by low expression of surface markers CD16, NKG2D, LDLR, and NKp30, and high expression rate of CD57.

In the present invention, the B cells may be preferably cells expressing CD19 or CD22, and the disease may be selected from the group consisting of tumors/cancer, lymphoma, non-Hodgkin's lymphoma (NHL), aggressive NHL, relapsed aggressive NHL, relapsed indolent NHL, refractory NHL, refractory indolent NHL, chronic lymphocytic leukemia (CLL), small lymphocytic lymphoma (SLL), leukemia, hairy cell leukemia (HCL), acute lymphocytic leukemia (ALL), Burkitt's lymphoma and mantle cell lymphoma.

In the present invention, the composition may include a therapeutic agent for a disease mediated by B cells, and the therapeutic agent may be present in a covalently bound state to an antibody specifically binding to CD19 or CD22, or may be administered in combination with the CD22-CAR immune effector cell or CD19xCD22-CAR immune effector cell of the present invention.

The therapeutic agent includes a small molecule drug, a peptidic drug, a toxin (for example, a cytotoxin), and the like.

The small molecule drug exhibits the pharmaceutical activity of a drug of interest and may generally be a compound having a molecular weight of about 800 Da or less or 2000 Da or less. An inorganic small molecule refers to a molecule that does not contain any carbon atoms, whereas an organic small molecule refers to a compound that contains at least one carbon atom.

The peptidic drug refers to an amino acid containing a polymeric compound, and includes naturally occurring and non-naturally occurring peptides, oligopeptides, cyclic peptides, polypeptides and proteins, as well as peptide mimetics. The peptide drug may be obtained by chemical synthesis or may be produced from a genetically encoded source (for example, a recombinant source). The peptide drug may have a molecular weight ranging from 200 Da to 10 kDa or more.

The toxin is preferably a cytotoxin, and non-limiting examples of the cytotoxin include ricin, abrin, diphtheria toxin, Pseudomonas exotoxin (for example, PE35, PE37, PE38, PE40, and the like), saporin, gelonin, pokeweed antiviral protein (PAP), botulinum toxin, bryodin, momordin and bouganin.

Further, the therapeutic agent may be an anticancer agent. The anticancer agent includes a non-peptidic (that is, non-protein-based) compound which reduces the proliferation of cancer cells and encompasses a cytotoxic drug and a cell proliferation inhibitor. Non-limiting examples of the anticancer agent include an alkylating agent, a nitrosourea, an antimetabolite, an anti-tumor antibiotic, a plant (vinca) alkaloid and a steroid hormone. A peptidic compound may also be used.

The humanized antibody, CD22-CAR immune effector cell or CD19xCD22-CAR immune effector cell specifically binding to CD22 in the pharmaceutical composition is the only active ingredient in the therapeutic or diagnostic composition, or can be used together with, for example, other antibody ingredients such as anti-T cells, and anti-IFNγ or anti-LPS antibodies, or other active ingredients including non-antibody ingredients such as xanthine.

The drug composition preferably includes a therapeutically effective amount of the antibody of the invention. As used herein, the term “therapeutically effective amount” refers to an amount of therapeutic agent required to treat, ameliorate or prevent a target disease or condition, or refers to an amount of therapeutic agent required to exhibit an appreciable therapeutic or prophylactic effect. For any antibody, the therapeutically effective dosage may be initially determined by cell culture assays or by animal models, usually rodents, rabbits, dogs, pigs or primates. Animal models may also be used to determine the appropriate concentration range and route of administration. This information may be used to determine useful dosages and routes for dosing in humans.

A precise effective amount for human patients may depend on the severity of the disease state, the general health status of the patient, the age, body weight and gender of the patient, the diet, the time of administration, the frequency of administration, the composition of the drug, the sensitivity of the reaction and the tolerance/response to the treatment. The amount may be determined by routine experimentation and is within the judgment of a clinician. In general, the effective dosage is 0.01 to 50 mg/kg, preferably 0.1 to 20 mg/kg, and more preferably about 15 mg/kg.

The composition may be administered individually to the patient, or may be administered in combination with other preparations, drugs or hormones.

The dosage at which the antibody of the present invention is administered depends on the nature of the condition to be treated, the grade of malignant lymphoma or leukemia, and whether the antibody is used to prevent disease or to treat an existing condition.

The frequency of administration depends on the half-life of the antibody molecule and the duration of the drug effect. When the antibody molecule has a short half-life (for example, 2 to 10 hours), it may be necessary to provide one or more doses per day. Alternatively, when the antibody molecule has a long half-life (for example, 2 to 15 days), it may be necessary to provide a dose once a day, once a week, or once every 1 or 2 months.

In addition, the pharmaceutical composition may contain a pharmaceutically acceptable carrier for administration of the antibody. The carrier itself should not cause the production of antibodies harmful to the individual to which the composition is administered, and should be non-toxic. Suitable carriers may be slowly metabolized macromolecules, such as proteins, polypeptides, liposomes, polysaccharides, polylactic acid, polyglycolic acid, amino acid polymers, amino acid copolymers and inactive viral particles.

Pharmaceutically acceptable salts are, for example, mineral acid salts such as hydrochloride, hydrobromide, phosphate and sulfate, or salts of organic acids such as acetic acid, propionic acid, malonic acid and benzoic acid may be used.

Pharmaceutically acceptable carriers in the therapeutic composition may additionally include liquids such as water, saline, glycerol and ethanol. Additionally, auxiliary substances such as wetting agents, emulsifying agents or pH buffering agents may be present in such compositions. The carrier may be formulated as tablets, pills, dragees, capsules, liquids, gels, syrups, slurries, and suspensions for ingestion of the pharmaceutical composition by the patient.

Preferred forms for administration include forms suitable for parenteral administration, for example by injection or infusion (for example, bolus injection or continuous infusion). When the product is for infusion or injection, it may take the form of a suspension, solution or emulsion in an oil or water-soluble excipient, which may include formulations such as suspending agents, preservatives, stabilizers and/or dispersants. Alternatively, the antibody molecule may be in anhydrous form, and may be reconstituted with an appropriate sterile solution before use.

Once formulated, the compositions of the present invention can be administered directly to a patient. Patients to be treated may be animals. However, it is preferred that the composition is tailored for administration to human patients.

The pharmaceutical composition of the present invention is not limited, but may be administered by any route including oral, intravenous, intramuscular, intraarterial, intramedullary, intrathecal, intraventricular, transdermal, transcutaneous (for example, see WO 98/20734), subcutaneous, intraperitoneal, intranasal, intestinal, topical, sublingual, vaginal or rectal routes. A hypospray may be used to administer the pharmaceutical composition of the present invention. Typically, the therapeutic composition may be prepared as an injectable material as a liquid solution or suspension. In addition, solid forms suitable for liquid excipient solutions or suspensions may be prepared prior to injection.

The direct delivery of the composition may generally be achieved by injection, subcutaneous injection, intraperitoneal injection, intravenous injection, or intramuscular injection, or may also be delivered to the interstitial space of the tissue. Furthermore, the composition may be administered to a wound site. Dosage treatment may be a single dosing schedule or multiple dosing schedules.

The active ingredient in the composition may be an antibody molecule. As such, it can be susceptible to degradation in the gastrointestinal tract. Therefore, when the composition is administered by a route using the gastrointestinal tract, the composition will need to contain an agent that protects the antibody from degradation but releases the antibody once absorbed from the gastrointestinal tract.

A complete discussion of pharmaceutically acceptable carriers is available in Remington's Pharmaceutical Sciences (Mack Publishing Company, NJ, 1991).

Hereinafter, preferred examples for helping the understanding of the present invention will be suggested. However, the following examples are provided only to more easily understand the present invention, and the contents of the present invention are not limited by the following examples.

### Example 1

### Preparation and Selection of Antibody Specifically Binding to CD22

In order to select an antibody specific for a CD22 peptide, a hybridoma that produces an antibody binding to CD22 was prepared to select the antibody.

First, spleen cells were excised by immunizing a CD22 protein (ACRObiosystems Inc., cat #CD2-H52H8, USA), and hybridoma cells were produced through cell fusion with mouse myeloma cells.

Mouse myeloma cells used for cell fusion cannot survive in the HAT medium because they do not have the Hypoxanthine Guanidine-Phosphoribosyl-Transforase (HGPRT) gene, but hybridomas can survive in the HAT medium by fusion with spleen cells. Since only hybridomas can be proliferated using the HAT medium, hybridomas are typically b in the HAT medium until the hybridomas are established.

A limiting dilution method was used to select a hybridoma that produces an antibody binding to CD22 from among the proliferated hybridomas. First, the number of cells was reduced to 1 cell or less per 96 wells, and then it was confirmed by ELISA whether an antibody obtained from a clone proliferated from one cell bound to CD22, and a clone binding to CD22 was selected. The above process was repeated three times to select hybridomas that produced the antibody binding to CD22. The antibody binding to CD22 was obtained in this manner.

The antibody is named 2G1 and these base sequences and amino acid sequences were analyzed. The sequence information on the heavy chain variable region and light chain variable region of each antibody according to the sequence analysis results is shown in the following Table 1, and the underlined part in Table 1 means a complementarity determining region (CDR).

TABLE 1
Sequence information of 2G1 antibody

| 2G1 | Sequence information | SEQ ID NO |
|---|---|---|
| Heavy chain | GFSLTSYDI | SEQ ID NO: 1 |
| variable region | | |
| CDR1 | | |
| Heavy chain | IWTGGGT | SEQ ID NO: 2 |
| variable region | | |
| CDR2 | | |
| Heavy chain | VPHYYGYAMDYW | SEQ ID NO: 3 |
| variable region | | |
| CDR3 | | |
| Light chain variable | QDINKY | SEQ ID NO: 4 |
| region CDR1 | | |
| Light chain variable | YTS | SEQ ID NO: 5 |
| site CDR2 | | |
| Light chain variable | LQYDNLLT | SEQ ID NO: 6 |
| site CDR3 | | |
| Heavy chain | EVQLQESGPGLVAPSQSLSITCTVSGFSLTS | SEQ ID NO: 7 |
| variable region | YDISWIRQPPGKGLEWLGVIWTGGGTNYN | |
| amino acid sequence | SAFMSRLSISKDNSKSQVFLKMNSLQTDDT | |
| | AIYYCVPHYYGYAMDYWGQGTSVTVSS | |
| Light chain variable | DIVLTQSPSSLSASLGGKVTITCKASQDINK | SEQ ID NO: 8 |
| region amino acid | YIAWYQHKPGKGPRLLIHYTSTLQPGIPSRF | |
| sequence | SGSGSGRDYSFSISNLEPEDIATYYCLQYDN | |
| | LLTFGAGTKLELK | |
| Heavy chain | GAGGTGCAGCTGCAGGAGTCAGGACCTG | SEQ ID NO: 9 |
| variable region base | GCCTGGTGGCGCCCTCACAGAGCCTGTCC | |
| sequence | ATTACCTGCACTGTCTCTGGGTTCTCATTA | |
| | ACCAGCTATGATATAAGCTGGATTCGCCA | |
| | GCCACCAGGAAAGGGTCTGGAGTGGCTTG | |
| | GAGTAATATGGACTGGTGGAGGCACAAAT | |
| | TATAATTCAGCTTTCATGTCCAGACTGAG | |
| | CATCAGCAAGGACAACTCCAAGAGCCAA | |
| | GTTTTCTTAAAAATGAACAGTCTGCAAAC | |
| | TGATGACACAGCCATATATTACTGTGTCC | |
| | CTCATTACTACGGCTATGCTATGGACTACT | |
| | GGGGTCAAGGAACCTCAGTCACCGTCTCC | |
| | TCA | |
| Light chain variable | GATATTGTGCTGACCCAGTCTCCATCCTCA | SEQ ID NO: 10 |
| region base | CTGTCTGCATCTCTGGGAGGCAAAGTCAC | |
| sequence | CATCACTTGCAAGGCAAGCCAAGACATTA | |
| | ACAAGTATATAGCTTGGTACCAACACAAG | |
| | CCTGGAAAAGGTCCTAGGCTGCTCATACA | |
| | TTACACATCTACATTACAGCCAGGCATCC | |
| | CATCAAGGTTCAGTGGAAGTGGGTCTGGG | |
| | AGAGATTATTCCTTCAGCATCAGCAACCT | |
| | GGAGCCTGAAGATATTGCAACTTATTATT | |
| | GTCTACAGTATGATAATCTGCTCACGTTC | |
| | GGTGCTGGGACCAAGCTGGAGCTGAAA | |

### Example 2

### Preparation of 2G1 Antibody-Based Humanized Antibody

A humanized antibody in which the 2G1 antibody selected in Example 1 was 5 changed to a structure corresponding to humans was prepared.

Specifically, mouse 2G1 antibodies were constructed into humanized antibodies by a CDR grafting method of replacing the CDR of a mouse antibody binding to CD22 with the CDR of a human antibody using a germline sequence of a human antibody as a frame. The humanized antibodies were named 2G1(V4) and 2G1(V12) and their amino acid sequences were analyzed. The sequence information on the heavy chain variable region and light chain variable region of the antibodies according to the sequence analysis results is shown in the following Tables 2 and 3, and the underlined parts in Tables 2 and 3 mean complementarity determining regions (CDRs).

TABLE 2
Sequence information of 2G1(V4) antibody

| 2G1-V4 | Sequence information | SEQ ID NO |
|---|---|---|
| Heavy chain | GFSLTSYDI | SEQ ID NO: 1 |
| variable region | | |
| CDR1 | | |
| Heavy chain | IWTGGGT | SEQ ID NO: 2 |
| variable region | | |
| CDR2 | | |
| Heavy chain | VPHYYGYAMDYW | SEQ ID NO: 3 |
| variable region | | |
| CDR3 | | |
| Light chain variable | QDINKY | SEQ ID NO: 4 |
| region CDR1 | | |
| Light chain variable | YTS | SEQ ID NO: 5 |
| region CDR2 | | |
| Light chain variable | LQYDNLLT | SEQ ID NO: 6 |
| region CDR3 | | |
| Heavy chain | EVQLQESGPGLVKPSQTLSLTCTVSGFSLTS | SEQ ID NO: 11 |
| variable region | YDISWIRQPPGKGLEWLGVIWTGGGTNYN | |
| amino acid sequence | SALKSRVTISKDNSKSQVSLKLSSVTAADTA | |
| | VYYCVPHYYGYAMDYWGQGTTVTVSS | |
| Light chain variable | EIVLTQSPATLSLSPGERATLSCRASQDINK | SEQ ID NO: 12 |
| region amino acid | YIAWYQQKPGQAPRLLIHYTSTRQTGIPARF | |
| sequence | SGSGSGRDYTLTISSLEPEDFAVYYCLQYDN | |
| | LLTFGGGTKLEIK | |
| Heavy chain | GAGGTGCAGCTGCAGGAGAGCGGCCCCG | SEQ ID NO: 13 |
| variable region base | GCCTGGTGAAGCCGAGCCAGACTCTTTCT | |
| sequence | CTGACCTGCACCGTGTCCGGCTTCTCTCTT | |
| | ACGAGCTACGACATCTCGTGGATCCGGCA | |
| | GCCGCCTGGGAAAGGCTTAGAGTGGCTAG | |
| | GGGTGATTTGGACCGGCGGGGGTACCAAC | |
| | TACAACTCCGCGCTCAAATCCCGCGTCAC | |
| | TATTTCTAAGGACAATTCCAAGAGCCAGG | |
| | TCTCGCTGAAGCTCTCGTCCGTGACCGCC | |
| | GCGGACACCGCAGTTTATTACTGCGTGCC | |
| | TCATTACTACGGCTACGCCATGGATTATT | |
| | GGGGCCAGGGCACCACAGTAACAGTCAG | |
| | CTCC | |
| Light chain variable | GAGATCGTGCTGACTCAGAGCCCGGCCAC | SEQ ID NO: 14 |
| region base | CCTTAGCCTGAGTCCAGGCGAGCGCGCTA | |
| sequence | CGTTGTCATGCCGAGCTTCCCAGGACATT | |
| | AACAAGTACATCGCGTGGTACCAGCAGAA | |
| | GCCCGGACAGGCCCCCCGCCTGCTCATCC | |
| | ACTACACCTCCACCCGCCAGACTGGCATC | |
| | CCTGCCAGGTTTTCAGGCTCCGGTTCTGGC | |
| | CGTGACTACACCCTGACCATCTCTAGTTTG | |
| | GAGCCCGAAGATTTCGCCGTGTACTACTG | |
| | TCTGCAATATGACAACCTGCTGACCTTCG | |
| | GAGGGGGTACCAAGCTGGAGATCAAG | |

TABLE 3
Sequence information of 2G1(V12) antibody

| 2G1-V12 | Sequence information | SEQ ID NO |
|---|---|---|
| Heavy chain | GFSLTSYDI | SEQ ID NO: 1 |
| variable region | | |
| CDR1 | | |
| Heavy chain | IWTGGGT | SEQ ID NO: 2 |
| variable region | | |
| CDR2 | | |
| Heavy chain | VPHYYGYAMDYW | SEQ ID NO: 3 |
| variable region | | |
| CDR3 | | |
| Light chain variable | QDINKY | SEQ ID NO: 4 |
| region CDR1 | | |
| Light chain variable | YTS | SEQ ID NO: 5 |
| region CDR2 | | |
| Light chain variable | LQYDNLLT | SEQ ID NO: 6 |
| region CDR3 | | |
| Heavy chain | EVQLKESGPVLVKPTETLTLTCTVSGFSLTS | SEQ ID NO: 15 |
| variable region | YDISWIRQPPGKALEWLGVIWTGGGTNYN | |
| amino acid sequence | SALKSRLTISKDNSKSQVVLTMTNMDPVDT | |
| | ATYYCVPHYYGYAMDYWGQGTTVTVSS | |
| Light chain variable | EIVLTQSPATLSLSPGERATLSCRASQDINK | SEQ ID NO: 16 |
| region amino acid | YIAWYQQKPGQAPRLLIHYTSTRQTGIPARF | |
| sequence | SGSGSGRDYTLTISSLEPEDFAVYYCLQYDN | |
| | LLTFGGGTKLEIK | |
| Heavy chain | GAGGTGCAGCTGAAGGAGAGCGGGCCGG | SEQ ID NO: 17 |
| variable region base | TGCTGGTGAAGCCTACCGAGACTCTGACC | |
| sequence | CTGACCTGCACTGTTTCCGGCTTCTCTCTG | |
| | ACGAGCTACGACATCAGTTGGATCCGCCA | |
| | GCCACCCGGCAAAGCGTTGGAATGGCTCG | |
| | GGGTAATTTGGACCGGTGGCGGGACCAAC | |
| | TACAACAGCGCGCTCAAATCGCGGCTAAC | |
| | CATCTCAAAGGACAACTCCAAGTCCCAAG | |
| | TGGTGTTAACTATGACAAATATGGATCCG | |
| | GTCGACACCGCTACCTATTACTGCGTGCC | |
| | TCATTACTACGGCTACGCCATGGATTATT | |
| | GGGGCCAGGGCACGACCGTGACCGTCTCC | |
| | AGT | |
| Light chain variable | GAGATCGTGTTGACCCAGAGCCCTGCCAC | SEQ ID NO: 18 |
| region base | GCTGAGCCTGTCCCCCGGGGAGCGCGCCA | |
| sequence | CTCTTTCGTGTAGGGCTTCCCAGGACATTA | |
| | ACAAGTACATCGCATGGTACCAGCAGAAG | |
| | CCCGGACAGGCCCCCCGCCTGCTCATCCA | |
| | CTACACATCCACCCGCCAGACAGGCATCC | |
| | CGGCTCGATTCTCTGGTTCTGGCAGCGGT | |
| | CGTGATTACACCCTTACTATTTCTTCCCTG | |
| | GAGCCAGAGGACTTTGCGGTGTACTACTG | |
| | CCTGCAGTATGACAACCTGCTGACCTTCG | |
| | GCGGAGGCACCAAGCTGGAGATCAAG | |

### Example 3

### Confirmation of Specificity of Selected Antibody to CD22

In the present invention, flow cytometry was performed to confirm the specificity of the 2G1 antibody (mouse) of Example 1 and the humanized 2G1(V4) and 2G1(V12) antibodies to CD22.

First, after 1×10⁶ non-cell lymphoma U2932 (B-cell lymphoma U2932 cell) cells expressing CD22 were reacted with 1 μg of the 2G1 antibody for 30 minutes, the surface was stained with a secondary antibody, and then measured by a flow cytometer.

A PE-conjugated anti-CD22 antibody (Biolegend Inc., cat #302506, USA) was used as a positive control, and a PE-conjugated goat anti-mouse IgG (Biolegend Inc., cat #405307, USA) was used as the secondary antibody.

As a result, as illustrated in FIG. 1, it was confirmed that all of the 2G1 antibody and humanized 2G1(V4) and 2G1(V12) antibodies specifically bound to cells expressing CD22.

### Example 4

### Construction of Chimeric Antigen Receptor (CD22-CAR) Expression Vector Targeting CD22

In the present invention, a lentiviral vector (CD22-CAR lentivirus) expressing the chimeric antigen receptor (CAR) targeting CD22 was constructed using the humanized 2G1(V4) and 2G1(V12) antibodies prepared in Example 2.

As illustrated in the schematic view of FIG. 3,
CAR DNA including: an EF1a promoter (SEQ ID NO: 29);
a polynucleotide (SEQ ID NO: 30) encoding a signal peptide;
a polynucleotide (2G1-V4 represented by SEQ ID NO: 26 or 2G1-V12 represented by SEQ ID NO: 28) encoding a CD22-binding domain;
a polynucleotide (SEQ ID NO: 31) encoding a CD8 hinge region;
a polynucleotide (SEQ ID NO: 32) encoding a transmembrane domain;
a polynucleotide (SEQ ID NO: 33) encoding 4-1BB (costimulatory domain);
a polynucleotide (SEQ ID NO: 34) encoding CD3ζ (intracellular signal transduction domain); and
a polynucleotide (SEQ ID NO: 35) encoding WPRE was synthesized in vitro and inserted into a 3^{rd} generation lentiviral vector.

The lentiviral vector was co-infected into HEK293FT cells with three vectors of pMDLg/pRRE (Addgene, cat ##12251) pMD2.G (Addgene, cat ##12259), and pRSV-Rev (Addgene, cat ##12253), and then a CD22-CAR lentiviral vector was produced. For co-infection, the three vectors and HEK293FT cells were cultured for 4 hours using a Lipofectamine 3000 transfection kit (Invitrogen, cat #L3000-015) and a medium of Opti-MEM+GlutaMAX (Gibco, cat #51985-034).

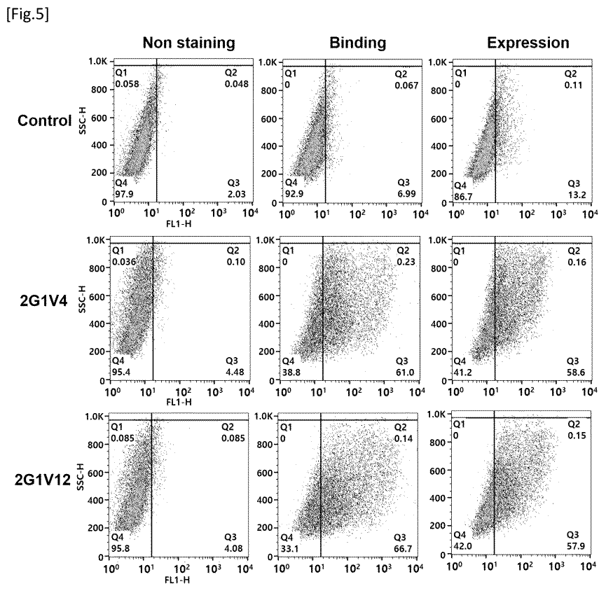

As a result of confirming whether a CAR specific for CD22 was expressed in HEK293FT transfected with the lentiviral vector (B of FIG. 4), as illustrated in FIG. 5, it was confirmed that an anti-CD22 antibody was normally expressed.

### Example 5

### Preparation of CD22-CAR-T Cells

In the present invention, humanized anti-CD22 antibody (2G1(V4) and 2G1(V12))-based CD22-CAR-T cells were respectively prepared by transforming T cells with the CD22-CAR lentiviral vector prepared in Example 4.

Specifically, as in the schematic view illustrated in FIG. 7, peripheral blood mononuclear cells (PBMCs) were isolated from blood, and then T cells were activated using T cell activation beads (Miltenyl Biotec, cat #130-091-441). CD22-CAR-T cells were prepared by transducing the CD22-CAR lentivirus prepared in Example 4 into the activated T cells.

The ability of CD22-CAR-T cells to bind to the CD22 peptide was confirmed by a flow cytometry method. After the CD22-CAR-T cells (2G1-V4 and 2G1-V12) were classified into CD22-CAR-T cells in which CD3, CD4, or CD8 was activated using anti-CD3, anti-CD4, and anti-CD8 antibodies, respectively, and then reacted with an FITC-CD22 peptide, fluorescence intensity was measured using an FACS instrument.

As a result, as illustrated in FIG. 8, it was confirmed that all the CD22-CAR-T cells in which CD3, CD4 or CD8 was activated bound to the CD22 peptide.

### Example 6

### Confirmation of Apoptotic Effect of CD22-CAR-T Cells on CD22-Expressing Cells

In the present invention, the apoptotic effect of humanized anti-CD22 antibody (2G1(V4) and 2G1(V12))-based CD22-CAR-T cells on target cells was confirmed.

As the target cells, K562 cells (human erythroleukemic cell line) which do not express CD22 and U2932 cells (B cell lymphoma) and NALM6 cells (human B cell precursor leukemia) expressing CD22 were used, and cultured for 8 hours by being mixed with CD22-CAR-T cells so as to have a ratio of 1:4, 1:2, 1:1, 1:0.5 and 1:0.25, respectively, and then luminescence (CytoTox-Glo Cytotoxicity Assay, Promega, cat. NO G9291) was measured. The degree of cell apoptosis was calculated from the measured values using the following Equation 1.

% Cytotoxicity=[(Experimental−Effector Spontaneous−Target Spontaneous)/(Target Maximum−Target Spontaneous)]×100 [Equation 1]
Experimental: Luminescence value derived from target cell and CAR-T cell composite culture medium
Effector Spontaneous: Luminescence value derived from CAR-T cell-only medium
Target Spontaneous: Luminescence value derived from target cell-only medium
Target Maximum: Luminescence value derived from 100% lysis (using lysis reagent) of target cells

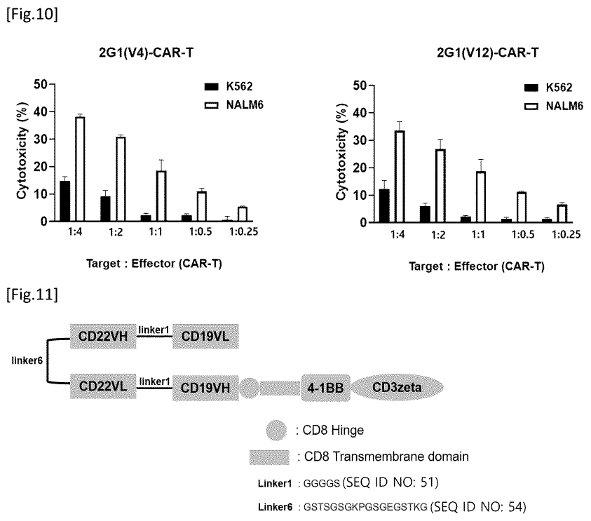

As a result, as illustrated in FIGS. 9 and 10, it was confirmed that the humanized anti-CD22 antibody (2G1(V4) and 2G1(V12))-based CD22-CAR-T cells specifically killed U2932 cells and NALM6 cells expressing CD22.

In the present invention, through the experiment, it was confirmed that humanized anti-CD22 antibody (2G1(V4) and 2G1(V12))-based CD22-CAR-T cells specifically killed diffuse large B-cell lymphoma-derived U2932 cells and acute lymphoblastic leukemia-derived NALM6 cells.

That is, the humanized anti-CD22 antibody (2G1(V4) and 2G1(V12))-based chimeric antigen receptor of the present invention and CAR-T cells using the same may be usefully used as a composition for preventing or treating a disease related to B cells or CD22 expression.

### Example 7

### Construction of Bispecific Chimeric Antigen Receptor Expression Vector Targeting CD19/CD22

A lentiviral vector (CD19xCD22-CAR lentivirus) expressing a bispecific chimeric antigen receptor targeting CD19 and CD22 was prepared in the same manner as in Example 4.

As illustrated in the schematic view of FIG. 11,
CAR DNA including: an EF1a promoter (SEQ ID NO: 29);
a polynucleotide (SEQ ID NO: 30) encoding a signal peptide;
a polynucleotide encoding a CD19/CD22-binding domain;
a polynucleotide (SEQ ID NO: 31) encoding a CD8 hinge region;
a polynucleotide (SEQ ID NO: 32) encoding a transmembrane domain;
a polynucleotide (SEQ ID NO: 33) encoding 4-1BB (costimulatory domain);
a polynucleotide (SEQ ID NO: 34) encoding CD37 (intracellular signal transduction domain); and
a polynucleotide (SEQ ID NO: 35) encoding WPRE was synthesized in vitro and inserted into a 3^{rd} generation lentiviral vector.

In the present invention, a known anti-CD19 antibody (FMC63) was used as a CD19-binding domain, and the 2G1 antibody, 2G1(V4) antibody and 2G1(V12) antibody of the present invention were used as a CD22-binding domain.

The CD19xCD22-binding domain may be linked in the order of a light chain variable region (CD19VL) of an antibody specifically binding to CD19—a heavy chain variable region (CD22VH) of an antibody specifically binding to CD22—a light chain variable region (CD22VL) of an antibody specifically binding to CD22—a heavy chain variable region (CD19VH) of an antibody specifically binding to CD19 (LoopCAR), and the sequence information on the polynucleotides encoding these is as follows:

CD19x 2G1: CD19VL represented by a base sequence of SEQ ID NO: 50—a linker (linker 1 in FIG. 11) represented by a base sequence of SEQ ID NO: 52-CD22VH represented by a base sequence of SEQ ID NO: 9—a linker (linker 6 in FIG. 11) represented by a base sequence of SEQ ID NO: 55—CD22VL represented by a base sequence of SEQ ID NO: 10—a linker (linker 1 in FIG. 1) represented by a base sequence of SEQ ID NO: 53—CD19VH represented by a base sequence of SEQ ID NO: 49;

CD19x2G1(V4): CD19VL represented by a base sequence of SEQ ID NO: 50—a linker (linker 1 in FIG. 11) represented by a base sequence of SEQ ID NO: 52-CD22VH represented by a base sequence of SEQ ID NO: 13—a linker (linker 6 in FIG. 11) represented by a base sequence of SEQ ID NO: 55—CD22VL represented by a base sequence of SEQ ID NO: 14—a linker (linker 1 in FIG. 1) represented by a base sequence of SEQ ID NO: 53—CD19VH represented by a base sequence of SEQ ID NO: 49;

CD19x2G1(V12): CD19VL represented by a base sequence of SEQ ID NO: 50—a linker (linker 1 in FIG. 11) represented by a base sequence of SEQ ID NO: 52—CD22VH represented by a base sequence of SEQ ID NO: 17—a linker (linker 6 in FIG. 11) represented by a base sequence of SEQ ID NO: 55—CD22VL represented by a base sequence of SEQ ID NO: 18—a linker (linker 1 in FIG. 1) represented by a base sequence of SEQ ID NO: 53-CD19VH represented by a base sequence of SEQ ID NO: 49.

The lentiviral vector was co-infected into HEK293FT cells with three vectors of pMDLg/pRRE (Addgene, cat ##12251) pMD2.G (Addgene, cat ##12259), and pRSV-Rev (Addgene, cat ##12253), and then a CD19/CD22-CAR lentiviral vector was produced. For co-infection, the three vectors and HEK293FT cells were cultured for 4 hours using a Lipofectamine 3000 transfection kit (Invitrogen, cat #L3000-015) and a medium of Opti-MEM+GlutaMAX (Gibco, cat #51985-034).

As a result of confirming whether a CAR specific for CD19xCD22 was expressed in HEK293FT transfected with the lentiviral vector, as illustrated in FIGS. 13 and 14, it was confirmed that an anti-CD19/anti-CD22 antibody was normally expressed.

### Example 8

### Preparation of CD19xCD22-CAR-T Cells

In the present invention, CD19x2G1, C19x2G1(V4) and CD19x2G1(V12), which are CD19xCD22-CAR-T cells, were respectively prepared by transforming T cells with the CD19xCD22-CAR lentiviral vector prepared in Example 7 in the same manner as in Example 5.

The ability of CD19xCD22-CAR-T cells to bind to the CD22 peptide was confirmed by a flow cytometry method. After the CD19xCD22-CAR-T cells (CD19x 2G1, CD19x 2G1(V4) and CD19 x2G1(V12)) were classified into CD19xCD22-CAR-T cells in which CD3, CD4, or CD8 was activated using anti-CD3, anti-CD4, and anti-CD8 antibodies, respectively, and then reacted with a PE-CD19 peptide and an FITC-CD22 peptide, fluorescence intensity was measured using a flow cytometer.

As a result, as illustrated in FIGS. 16a-16c, it was confirmed that all the CD19xCD22-CAR-T cells in which CD3, CD4 or CD8 was activated bound to the CD19 peptide and the CD22 peptide.

### Example 9

Confirmation of Activation of CD19xCD22-CAR-T Cells in CD22-Expressing Cells

In the present invention, in order to confirm whether the CD19xCD22-CAR-T cells prepared in Example 8 were specifically activated by CD22-expressing cells, the expression level of IFNγ by CD19xCD22-CAR-T cells was confirmed in the presence of target cells.

As the target cells, K562 cells (ATCC, cat #CCL-243) which do not express CD19 and CD22 and NALM6 cells (human B cell precursor leukemia) expressing CD19 and CD22 were used, and CD19/CD22-CAR-T cells and the target cells were reacted at a ratio of 2:1, 1:1, 0.5:1, and 0.25:1 for a certain period of time, and then stained with a surface & intra antibody, and measured by a flow cytometer (INF-r, CD4, and CD8 staining).

As a result, as illustrated in FIGS. 17A to 17C, it was confirmed that T cells were not activated in K562 cells that do not express CD19 and CD22, whereas T cells were activated in the presence of NALM6 cells expressing CD19 and CD22, thereby increasing the expression of IFNγ.

### Example 10

Confirmation of Apoptotic Effect of CD19xCD22 cells on CD22 or CD19 Expression Cells

In the present invention, the apoptotic effect of humanized anti-CD22 antibody (2G1(V4) and 2G1(V12))-based CD19xCD22-CAR-T cells on target cells was confirmed.

As the target cells, K562 cells (human erythroleukemic cell line) which do not express CD22 and NALM6 cells (human B cell precursor leukemia) expressing CD19 and CD22 were used, and cultured for 8 hours by being mixed with CD19xCD22-CAR-T cells so as to have a ratio of 1:4, 1:2, 1:1, 1:0.5 and 1:0.25, respectively, and then luminescence (CytoTox-Glo Cytotoxicity Assay, Promega, cat #G9291) was measured. The degree of cell apoptosis was calculated from the measured values using Equation 1 in Example 6.

As a result, as illustrated in FIG. 18a, it was confirmed that mouse-derived 2G1-based CD19x2G1 CAR-T, humanized antibody-based CD19x2G1(V4) CAR-T of 2G1 and other humanized antibody-based CD19x2G1(V12) CAR-T cells of 2G1 specifically killed NALM6 cells expressing CD22 and CD19.

In the present invention, K562 cells (ATCC, cat #CCL-243) which do not express CD19 and CD22 and three cells K562/CD19+, K562/CD22+, and K562/CD19+/CD22+ expressing CD19 or CD22 or CD19xCD22 were constructed, and the apoptotic effect of humanized antibody-based CD19x2G1(V4) CAR-T cells on the target cells was confirmed. As a result, as illustrated in FIG. 18b, it was confirmed that CD19x2G1(V4) CAR-T cells specifically killed cells expressing CD22 or CD19 or

CD19/CD22.

### Example 11

### Confirmation of Antitumor Effects of CD19/CD22-CAR-T Cells in Animal Model

In the present invention, the antitumor effects of CD19 x CD22-CAR-T cells were confirmed using a mouse model in which tumor cells were xenografted.

After 1×10⁶ NALM6/Luc cells were injected into 9-week-old NOD/SCID mice by intravenous injection, 0.5×10⁶ (low), 1×10⁶ (medium), and 5×10⁶ (high) CD19xCD22-CAR-T cells were injected by intravenous injection on day 5, and 1.25×10⁷

Palivizumab CAR-T cells as a positive control were injected. After cell injection, the antitumor effects by suppressing the proliferation of the NALM6/Luc cells were observed by capturing the luminescence expressed in the NAML6/Luc cells ex vivo by IVIS SpectrumCT on days 3, 8, 15, 22, and 29.

As a result, as illustrated in FIG. 19a, it was confirmed that the antitumor effects of the CD19xCD22-CAR-T cells of the present invention were excellent compared to those of palivizumab-CAR-T cells derived from a scFv of the antibody palivizumab binding to the SV40 virus used as a positive control and an animal group into which CAR-T was not injected, and it was confirmed that when CD19xCD22-CAR-T cells were treated at 5×10⁶ or more, most of the tumor cells were killed, and thus no tumor cells expressing luminescence were observed until day 29 after CAR-T injection.

As a result of showing these results as the survival rate curve in FIG. 19b, it was confirmed that all the mice injected with CD19xCD22-CAR-T at 1×10⁶ (medium) and 5×10⁶ (high) survived until day 29.

### Example 12

### Establishment of Optimal Treatment Conditions of Dexamethasone for Promoting NK Cell Differentiation Induction

In this example, dexamethasone was used to promote the induction of differentiation of PBMCs into NK cells, and the NK cell growth curve according to the treatment concentration, exposure time, and treatment time of dexamethasone was confirmed to establish optimal treatment conditions.

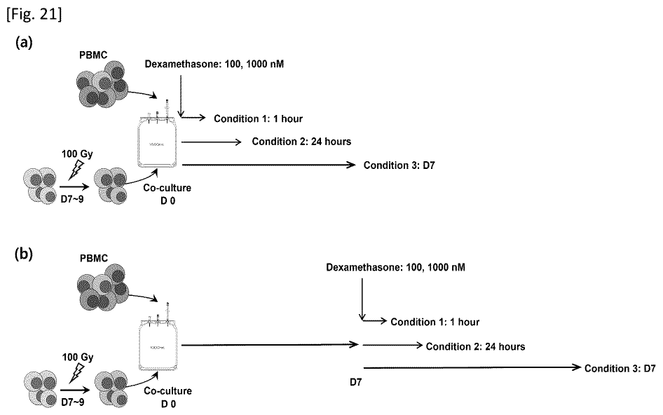

Dexamethasone was treated by the method shown in the schematic diagram of FIG. 21. Specifically, the timing of dexamethasone treatment was arbitrarily designated as the first day of culture 0 and the last day of culture 7 in the initial NK activation stage, and the treatment concentration was arbitrarily designated as 100 nM and 1000 nM within the range of 50 to 1000 nM. The exposure time was arbitrarily set to three conditions: 1 hour, 24 hours, and culturing for 7 days without washing. The initial culture conditions were co-cultured with PBMCs at 1.5×10⁶ cells/mL and K562 at 5×10⁵ cells/mL. The medium used was serum-free medium.

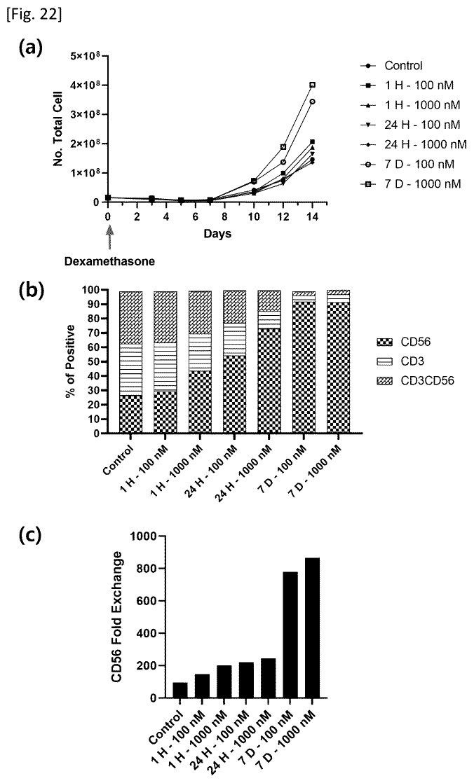

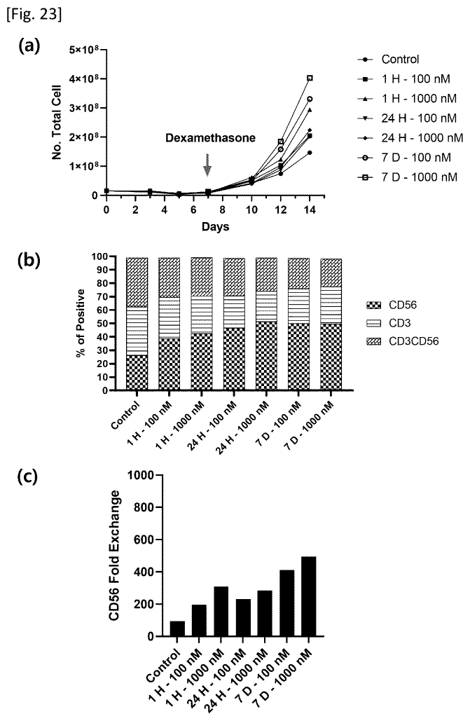

As a result, as shown in FIGS. 22a and 23b, when 1000 nM dexemthasone was treated on day 0 of culture and cultured for 7 days without washing, the final NK cell concentration on day 14 of culture was confirmed to be the highest.

On the 14th day of culture, the PBMC cell population was confirmed using a flow cytometer. As shown in FIG. 22b and FIG. 23b, in general, the number of NK cells was confirmed to be higher in the case of dexamethasone treatment on the 0th day of culture than in the case of treatment on the 7th day of culture. In particular, when comparing the groups that showed high cell growth in the growth curve, the conditions exposed on the 7th day of culture showed about 50% of NK cells, whereas the conditions exposed on the 0th day of culture showed about 90% of NK cells, confirming a high number of NK cells.

The NK cell expansion fold was also confirmed to be the highest at approximately 700-fold or more when dexamethasone was treated on day 0 of culture and cultured for 7 days without washing.

In summary, it was confirmed that dexamethasone can increase the number of NK cells and decrease the proportion of other T cells, etc., when treated at the early stage of culture and with a longer exposure time, and can also help cell growth.

### Example 13

### Establishment of Optimal Conditions for Enhancing CAR Expression Rate of CD19xCD22-CAR-NK Cells—Confirmation of Cell Phenotype and CAR Expression

PBMCs were separated from blood aliquots obtained from leukapheresis of healthy donors by Ficoll-paque density gradient centrifugation, and then cryopreserved. The cryopreserved PBMCs were thawed and co-cultured with K562 feeder cells irradiated with 100 Gy at a ratio of 3:1 to induce differentiation into NK cells. On days 3, 5, 7, and 10 of inducing NK differentiation, the cells were harvested after co-culture for transduction, centrifuged at 450 ×g for 5 min at RT, and then treated with 5 MOI of the CD19xCD22-CAR lentiviral vector manufactured in <Example 1>. After 24 hours, NK cells were harvested and replaced with NK culture medium supplemented with IL-2 and IL-15, and additionally cultured for a total of 14 days. On the final culture day, NK cells were treated with CD56 (APC), CD3 (Pacific blue), and CD19 (PE) recombinant proteins, and the cell phenotype and CAR expression were analyzed using a flow cytometer (Cytek).

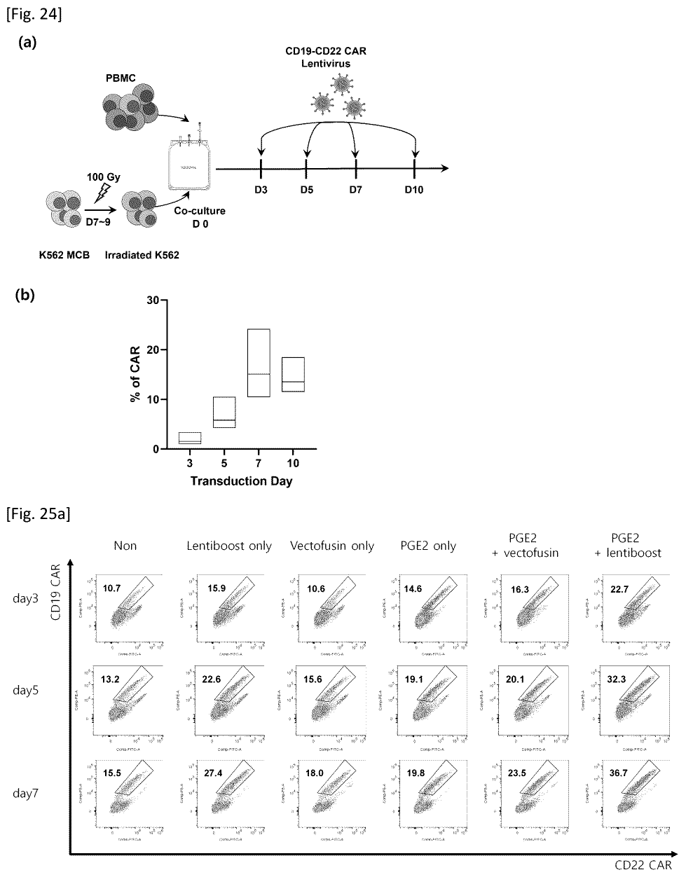

As a result, as shown in FIG. 24, it was confirmed that the CAR expression rate was the best when the CD19xCD22-CAR lentiviral vector was transduced on days 7 and 10 of NK differentiation induction.

### Example 14

### Establishment of Optimal Conditions for Enhancing CAR Expression Rate of CD19xCD22-CAR-NK Cells—Confirmation of CAR Expression According to Additives During Transduction

PBMCs were separated from blood aliquots obtained from leukapheresis of healthy donors by Ficoll-paque density gradient centrifugation, and then cryopreserved. The cryopreserved PBMCs were thawed and co-cultured with K562 feeder cells irradiated with 100 Gy at a ratio of 3:1 to induce differentiation into NK cells. The co-culture was performed for a total of 7 days, and 200 ml of culture medium was added on days 3 and 5 for a total of 600 ml of culture. The cells co-cultured for 7 days were centrifuged at 1,500 rpm and 18° C., and CD3 T cells were removed using a positive selection method (MACS, CD3 depletion kit). After obtaining activated NK cells, 2×10⁷ cells were placed in T75 flasks, treated with additives, and divided into the following treatment groups: (1) 1:100 Lentiboost® (Poloxamer synperonic F108; Non-ionic, Amphiphilic poloxamer synperonic F108, Sirion Bioteck) only treatment group, (2) 10 μg/mL Vectofusin (Miltenyi Biotec) only treatment group, (3) 10 μM PGE2 (Sigma) only treatment group, (4) 10 μM PGE2 +10 μg/mL Vectofusin combined treatment group, and (5) 10 μM PGE2 +1:100 Lentiboost® combined treatment group.

CD19xCD22-CAR lentiviral vector was diluted to the same culture volume as NK cells and treated with 5 MOI. After 24 hours, NK cells were harvested and replaced with NK cell culture medium supplemented with IL-2 and IL-15, and further cultured for a total of 7 days to produce CD19xCD22-CAR-NK cells.

On days 3, 5, and the final day of culture (day 7), the cells were diluted with NK culture medium to a concentration of 5×10⁵ cells/mL, and then treated with CD19-FITC, CD22-PE (AcroBio systems) recombinant proteins. CAR-positive cells were analyzed using a flow cytometer (Cytek). Statistical analysis was performed using the student-t Test in GraphPad Prism software (P value, 0.001>***).

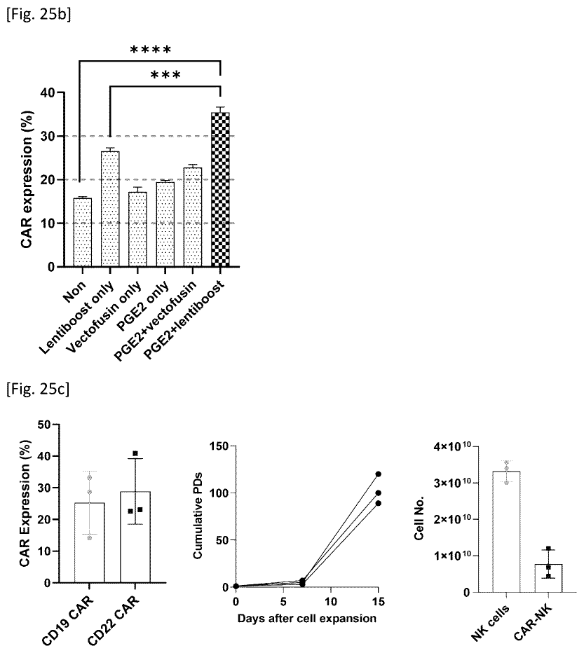

The results of the CAR positive cell analysis and the analysis of the % of CAR positive cells 7 days after transduction are shown in FIGS. 25a and 25b, and it was confirmed that the CAR expression rate was the highest in the PGE2 +Lentiboost® combination treatment group.

CD19xCD22-CAR-NK cells were prepared in the same manner using PBMCs from three healthy donors under the PGE2 +Lentiboost® complex treatment conditions established above. CAR expression and proliferation rates were confirmed on the final day of culture, and CAR expression and CD19xCD22-CAR-NK cell proliferation were confirmed (FIG. 25c). Non-transduced normal NK cells were used as a negative control.

### Example 15

Establishment of Optimal Conditions for Enhancing CAR Expression Rate of CD19xCD22-CAR-NK Cells-Establishment of MOI To enhance the CAR expression rate of CD19xCD22-CAR-NK cells and to confirm CAR expression according to MOI of CD19xCD22-CAR lentiviral vector, CD19xCD22-CAR lentiviral vector was transduced at 0 MOI, 0.5 MOI, 1 MOI, 2 MOI, 5 MOI, and 10 MOI on day 7 of NK differentiation induction, respectively. 24 hours after transduction, NK cells were harvested and replaced with NK cell culture medium supplemented with IL-2 and IL-15, and further cultured for 7 days. 7 days after transduction, CD19xCD22-CAR-NK cells transduced at different MOls were harvested, treated with CD56 (APC), CD3 (Pacific blue), CD19 (PE) recombinant protein, and CD22 (FITC) recombinant protein, and CAR expression was analyzed using a flow cytometer (Cytek), respectively. Cell viability was determined using a cell counting device.

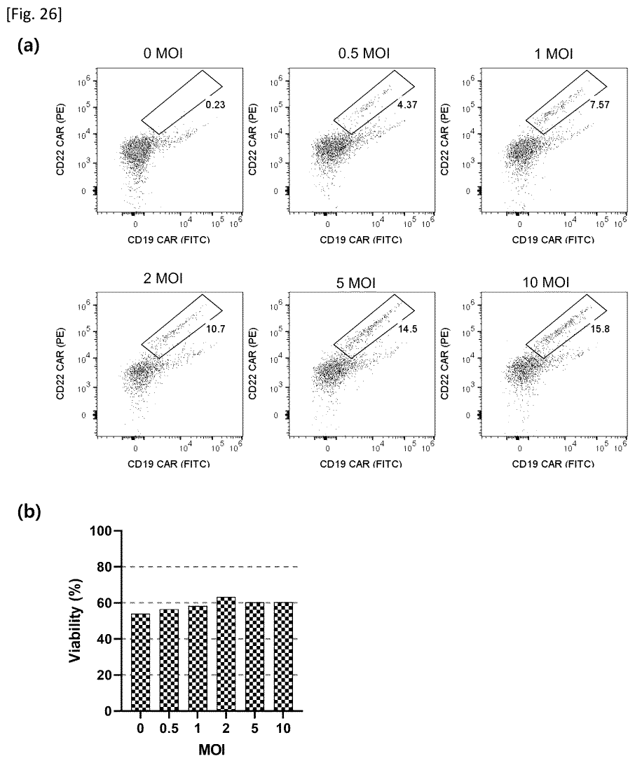

As a result, as shown in FIG. 26, it was shown that the transduction efficiency was close to the maximum under the 5 MOI condition.

### Example 16

### Preparation of CD19xCD22-CAR-NK Cells Using the Present Established Culture Process

In this example, based on the experimental results of the above example,
(1) dexamethasone was treated at the beginning (day 0) of NK differentiation induction, and differentiation induction was performed for 5 to 10 days (preferably 7 days) in a medium containing dexamethasone,
(2) NK cells induced for differentiation in the above (1) were treated with 3 to 7 MOI (preferably 5 MOI) when transducing CD19xCD22-CAR lentiviral vector,
(3) a culture process was established that additionally included PGE2 and LentiBOOST to increase the expression rate of chimeric antigen receptor (CAR) during transduction.

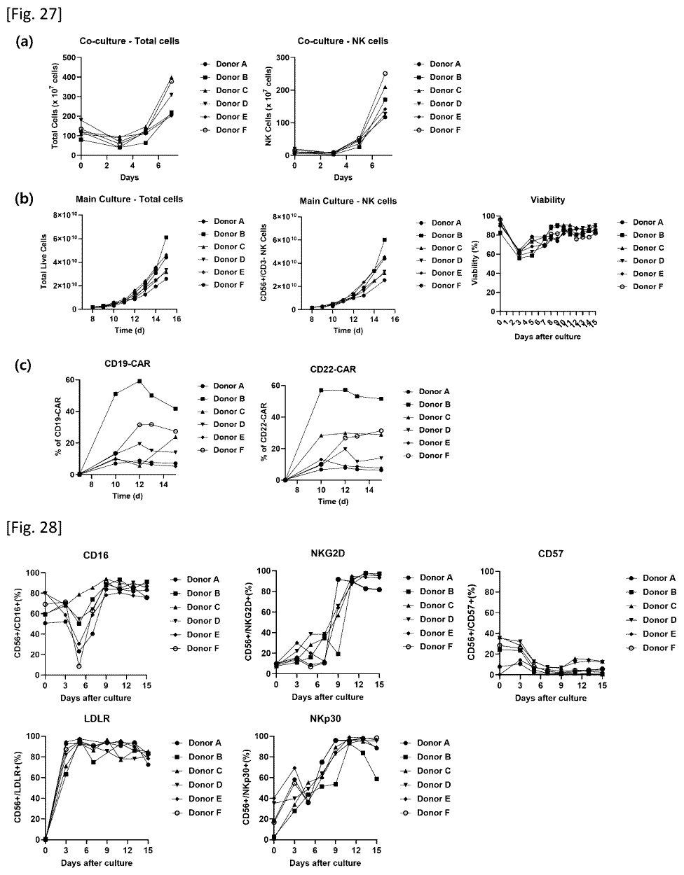

In this example, the present culture process established above was applied to PBMCs isolated from six healthy donors (donors A to F) to induce differentiation of NK cells, and the growth rate of NK cells was shown in a of FIG. 27.

CD19xCD22-CAR lentiviral vector was transduced into the NK cells cultured above to produce CD19xCD22-CAR-NK cells. Then, on the 8th day after transduction, the cells were inoculated into a 10 L bioreactor (Bioreactor; Eppendorf) and mass culture was performed for a total of 8 days. After inoculation into the bioreactor, NK culture medium was added three times at 1.6 L intervals every 2 days. The culture conditions were maintained at 37° C., 5% CO₂, and 100 rpm, and the pH was maintained at 6.5 to 7.2.

During the culture period, 3 mL of samples were taken daily, and the cell count and cell viability were measured using an automatic cell counter. On the final day of culture, a final 350 ml of cell culture solution was recovered through a continuous centrifugation process using UniFuge equipment. The recovered culture bag was washed a total of 5 times with PlasmaLyte A 148 strain, and CS10 and Human Serum Albumin were added and frozen. During mass culture, the CAR expression rate was measured by measuring the fluorescence signal using a flow cytometer after treating the cells with CD19-FITC, CD22-PE (AcroBio systems) recombinant proteins.

The growth rate and CAR expression rate of CD19xCD22-CAR-NK cells were shown in b and c FIG. 27, respectively.

In addition, from day 0 to day 15 of culture, the cells were stained using surface markers CD16-FITC (Biolegend), NKG2D-APC (BD Pharmingen), CD57-BV785(Biolegend), LDLR-PE (Biolegend), and NKp30-APC/Cy7 (BD Pharmingen), and analyzed using a flow cytometer. The results are shown in FIG. 28.

Examining the results in FIGS. 27 and 28, when CD19xCD22-CAR-NK cells were manufactured by applying the culture process established in the present invention, it was found that there was a significant difference in the CAR expression rate and the expression rate of surface markers depending on the type of donated PBMC. Accordingly, in the present invention, we aimed to establish suitable PBMC conditions for the production of CD19xCD22-CAR-NK cells.

### Example 17

### Establishment of PBMC Selection Criteria Suitable for CD19xCD22-CAR-NK Cell Production

In this example, based on the results of the above <Example 6>, PBMC selection criteria suitable for CD19xCD22-CAR-NK cell production was established.

First, PBMCs isolated from each donor were stained with CD16-FITC (Biolegend), NKG2D-APC (BD Pharmingen), CD57-BV785 (Biolegend), LDLR-PE (Biolegend), and NKp30-APC/Cy7 (BD Pharmingen), and then analyzed using a flow cytometer to analyze the expression pattern of each gene to analyze the characteristics of the donors.

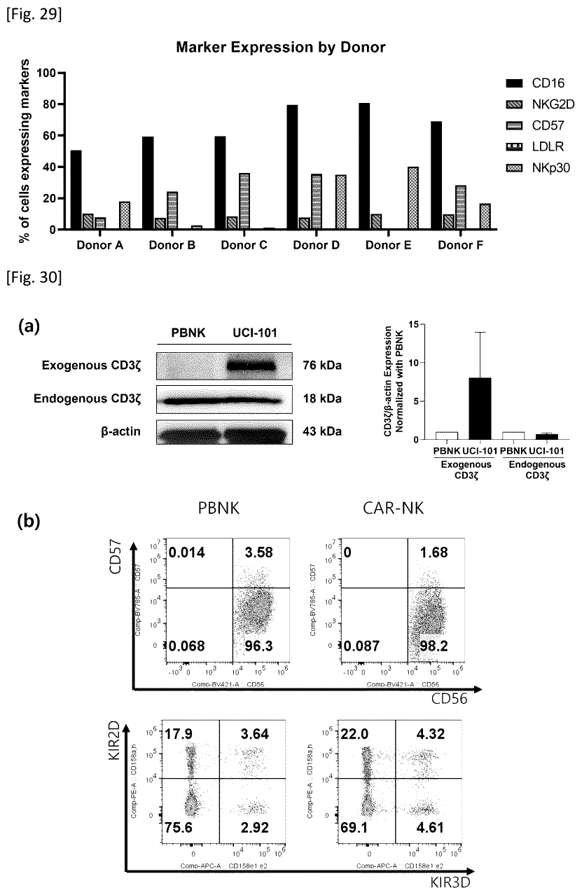

As a result, it was confirmed that PBMCs isolated from Donor B in the process of the above <Example 6> had higher CAR gene transfer efficiency and higher NK cell proliferation rate than PBMCs isolated from other donors (FIGS. 27 and 28), and showed a unique gene expression pattern as shown in FIG. 29 and Table 4 below, unlike other donors.

TABLE 4
Analysis results of surface markers of Donor B

| Selection marker | Selection criteria (%) | Summary |
|---|---|---|
| CD16 | 70% or less | 70% ≥ CD16 |
| NKG2D | less than 10% | 10% > NKG2D |
| CD57 | 30% or less | 30% ≥ CD57 |
| LDLR | 0.1% or more | 0.1% ≤ LDLR |
| NKp30 | less than 10% | 10% > NKp30 |

Based on Table 4 above, the selection criteria for donors with high CAR gene transfer efficiency was established as follows: CD16: 70% or less, NKG2D: less than 10%, CD57: 30% or less, LDLR: 0.1% or more, and NKp30: less than 10%.

TABLE 5
Analyze surface markers for each donor and verify they meet selection criteria

| | Donor A | | Donor B | | Donor C | | Donor D | | Donor E | | Donor F | |
|---|---|---|---|---|---|---|---|---|---|---|---|---|
| | Expres- | | Expres- | | Expres- | | Expres- | | Expres- | | Expres- | |
| | sion | Suit- | sion | Suit- | sion | Suit- | sion | Suit- | sion | Suit- | sion | Suit- |
| Item | rate | ability | rate | ability | rate | ability | rate | ability | rate | ability | rate | ability |
| CD16 | 50.6% | Suitable | 59.3% | Suitable | 59.7% | Suitable | 79.5% | Unsuitable | 80.8% | Unsuitable | 69.1% | Suitable |
| NKG2D | 10.3% | Unsuitable | 7.55% | Suitable | 8.54% | Suitable | 7.74% | Suitable | 10.1% | Unsuitable | 9.8% | Suitable |
| CD57 | 7.87% | Suitable | 24.3% | Suitable | 36.3% | Unsuitable | 35.5% | Unsuitable | 0.18% | Suitable | 28.4% | Suitable |
| LDLR | 0% | Unsuitable | 0.5% | Suitable | 0% | Unsuitable | 0% | Unsuitable | 0.21% | Suitable | 0% | Unsuitable |
| NKp30 | 18% | Unsuitable | 2.77% | Suitable | 1.3% | Suitable | 35% | Unsuitable | 40.1% | Unsuitable | 16.6% | Unsuitable |
| Whether | No | | Yes | | No | | No | | No | | No | |
| Selection | | | | | | | | | | | | |
| conditions | | | | | | | | | | | | |
| were met | | | | | | | | | | | | |
| Results of NK | Donor A | | Donor B | | Donor C | | Donor D | | Donor E | | Donor F | |
| production | | | | | | | | | | | | |
| CD19 | 6.95% | | 51.0% | | 10.3% | | 12.9% | | 9.76% | | 13.4% | |
| expression | | | | | | | | | | | | |
| (TD 10th day) | | | | | | | | | | | | |
| CD22 | 6.67% | | 57.0% | | 28.3% | | 9.63% | | 13.1% | | 10.0% | |
| expression | | | | | | | | | | | | |
| (TD 10th day) | | | | | | | | | | | | |
| NK cell count | 4.392 × 10¹⁰ | | 6.023 × 10¹⁰ | | 3.134 × 10¹⁰ | | 3.299 × 10¹⁰ | | 4.353 × 10¹⁰ | | 2.532 × 10¹⁰ | |
| (15th day of | | | | | | | | | | | | |
| incubation) | | | | | | | | | | | | |

The selection criteria for donors established above were applied to PBMCs from each donor to confirm whether they met the selection criteria, and only Donor B was found to be suitable, with the other donors having very low levels of CAR expression and CAR-NK cell counts.

### Example 18

### Analysis of CD19xCD22-CAR-NK Cells (UCI-101) Manufactured Under Optimal Conditions

In this example, CD19xCD22-CAR-NK cells were produced as in <Example 6> using PBMC of Donor B selected under the conditions of <Example 7>, and named “UCI-101”.

To confirm the characteristics of UCI-101, the phenotype of PBNK (periopheral blood NK cell, control) and UCI-101 (CAR-NK) was analyzed.

First, Western blotting was performed to measure the expression levels of exogenous and endogenous CD3ζ. Western blotting was performed according to standard procedures and transferred to PVDF (Bio-rad) membranes. The primary antibodies used were anti-CD3ζ (Cell Signalling Technology, 1:1000) and anti-β-actin-HRP (Santa cruz, 1:1000). Anti-rabbit IgG (Santa cruz, 1:5000) HRP-conjugated antibody was used as the secondary antibody, and detection was performed using ChemiDoc Imaging System (Biorad, BR17001401) using ECL reagent (Super Signal West Pico, Thermo Scientific). The bar graph represented the mean±standard deviation of the quantification results of duplicate Western blot gel images using the Prism program. The gel images were quantified using the ImageJ program and normalized based on the β-actin quantification value. Afterwards, the PBNK result value was set to 1 and the expression level of CAR-NK was compared and analyzed.

As a result, as shown in a of FIG. 30, exogenous CD3ζ expression increased in UCI-101, indicating that CAR was normally expressed on the surface of NK cells.

Next, to determine the degree of aging of UCI-101 depending on the presence or absence of transduction, PBNK without lentiviral vector treatment was prepared using the same culture method as PBMC of the same healthy donor as UCI-101. Then, UCI-101 and PBNK were stained with CD57-BV785 (Biolegend), CD56-BV421 (Biolegend), CD158a.h-APC (BD Pharmingen), and CD158e1.e2 (BD Pharmingen) fluorescent antibodies and analyzed using a flow cytometer.

As a result, as shown in b of FIG. 30, it was confirmed that aging of UCI-101 due to transduction was not promoted.

### Example 19

### Confirmation of the Killing Effect of CD19xCD22-CAR-NK Cells (UCI-101) on CD22 or CD19 Expressing Cells

The CAR expression rate and cytotoxicity against target cells (Raji cells and Daudi cells) of CD19xCD22-CAR-NK cells (UCI-101) manufactured by the preparation method of the present invention were confirmed.

Raji cells, a Burkit lymphoma-derived cell, and Daudi cells, a human B-cell lymphoma cell, are representative B cell lines known to express CD19 and CD22 antigens.

First, in order to confirm the CAR expression rate of UCI-101 manufactured in the above <Example 8>, CD19-FITC, CD22-PE (AcroBio systems) recombinant proteins were treated to the cells, and then the fluorescence signal was measured using a flow cytometer. The target cells, Raji cells and Daudi cells, were cultured for more than a week and used after a stabilization period. The target cells were subjected to CTV labeling (100 nM) in a 37° C. incubator for 20 minutes at a concentration of 1×10⁷ cells/mL, and then washed twice or more and co-cultured with thawed PBNK or UCI-101 for 4 hours.

The cells were cultured in 24-well plates at a ratio of 5×10⁵ cells, calculated based on the total number of cells, so that the ratio of UCI-101 to target cells (E:T Ratio) was 9:1, 3:1, and 1:1. After 4 hours, the cells were harvested, stained with PI, and analyzed by flow cytometry. The degree of PI expression within the CTV positive gate was analyzed to measure the degree of cytotoxicity of the target cells using the following mathematical formula 1.

After 4 hours, the cells were recovered, PI stained, and flow cytometry was performed. The level of PI expression was analyzed within the CTV positive gate, and the degree of cytotoxicity of the target cells was measured using Equation 1 in Example 6.

Next, the amount of TNF-alpha and IFN-gamma cytokines secreted from the supernatant obtained during cell recovery was measured using ELISA (Enzyme Linked Immunosorbent assay). ELISA was performed according to standard procedures (R&D systems), and the final absorbance was analyzed using a Multiplate Reader (Molecular Devices, spectramax id3 system).

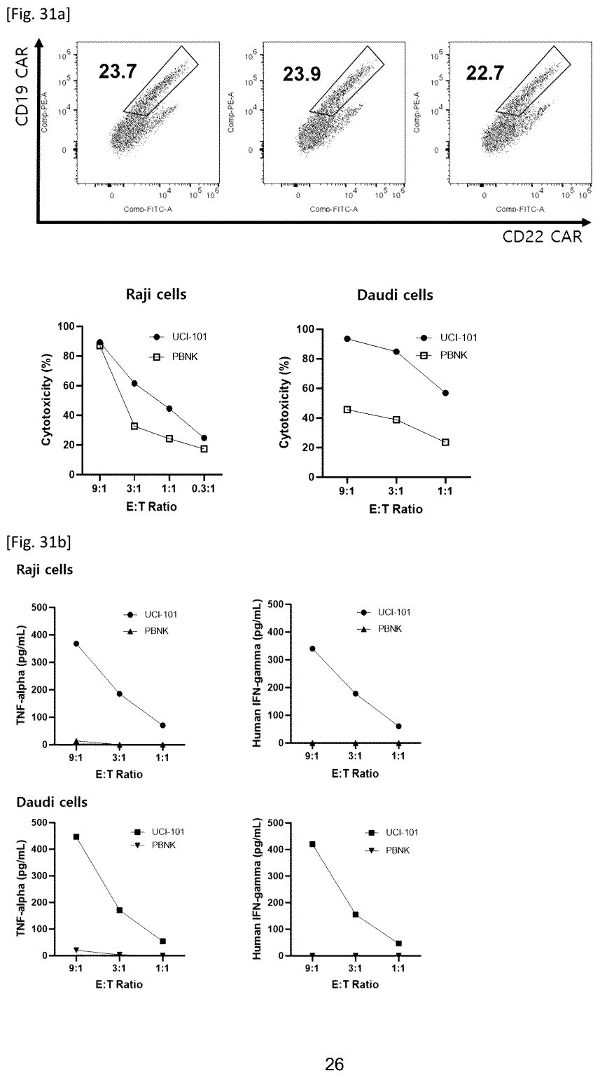

As a result, as shown in FIG. 31b, it was confirmed that UCI-101 secretes cytokines that play an important role in cell killing ability and anticancer activity.

### Example 20

### Confirmation of Antitumor Effect of CD19xCD22-CAR-NK Cells (UCI-101) in Animal Models

In this example, the antitumor effect of CD19xCD22-CAR-NK cells (UCI-101) was confirmed in the U2932 blood cancer animal model.

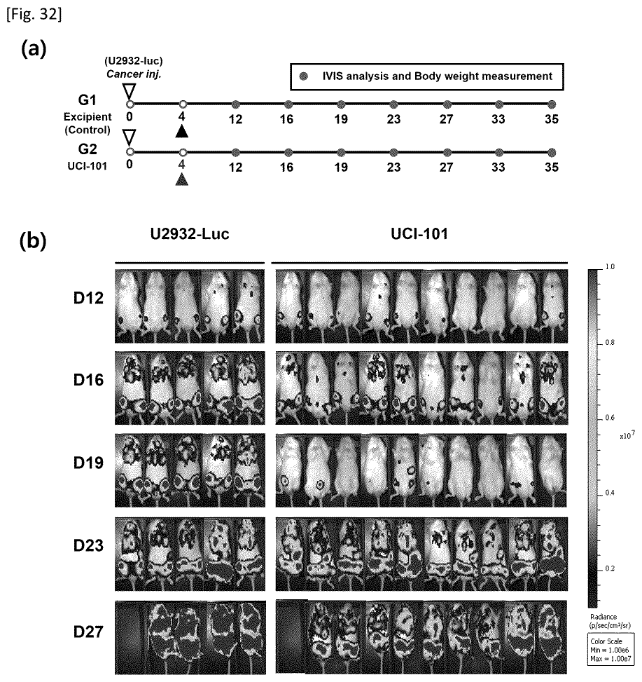

Male NOG mice (NOD.Cg-Prkdcscidll2rgtm1Sug/Jic) were supplied by Japan CIEM, acclimatized for 1 week, and then used for animal testing. Animal testing was performed under the approval of the Animal Ethics Committee, and the test was outsourced to Samda Bio Co., Ltd. The acclimatized mice were intravenously injected with 4×10⁶ cells per mouse of the U2932-Luc cell line. The time point of injection of the U2932-Luc cell line was set as day 0, and 4 days later, CD19xCD22-CAR-NK cells (UCI-101) were intravenously injected per mouse at 5×10⁶ cells based on CAR-positive cells (a of FIG. 32). Images were measured on days 12, 16, 19, 23, and 27 using IVIS (Perkin Elmer) imaging equipment. The substrate used at this time was Luciferin (Perkin Elmer), which was measured 5 minutes after intraperitoneal injection. For each measurement, 3 to 5 mice were anesthetized and then measured.

After administration of U2932-Luc cells, IVIS imaging results were confirmed on days 12, 16, 19, 23, and 27. Luminescence values were imaged using Pseudocolor. The color bar represents the relative Luciferase activity in p/sec/cm2/sr, and the minimum value is 1×10⁶ and the maximum value is 1×10⁷.

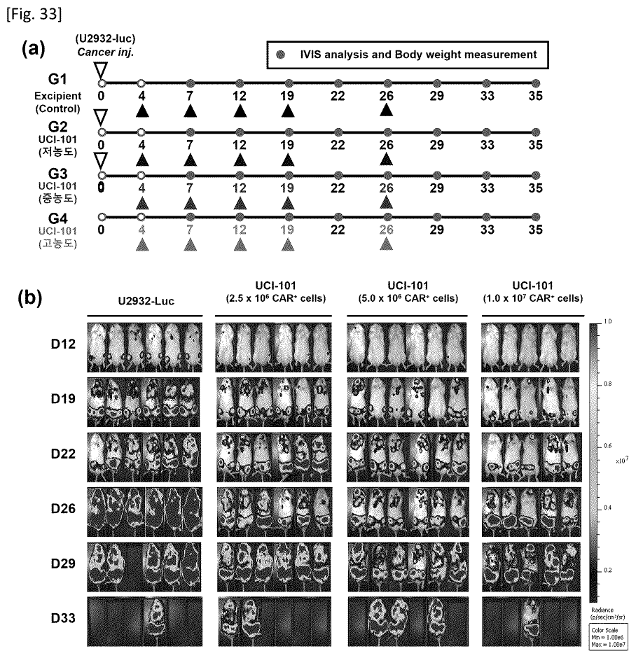

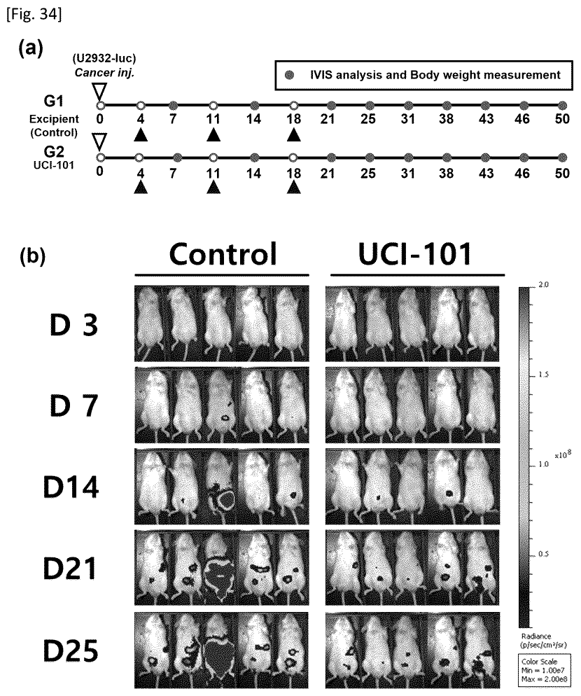

Next, in order to establish the administration concentration and administration cycle of UCI-101, UCI-101 was administered as shown in the schematic diagrams of a of FIG. 33 and a of FIG. 34.

Specifically, to establish the administration concentration of UCI-101, 4, 7, 12, and 19 days after injection of the U2932-Luc cell line into mice, CD19xCD22-CAR-NK cells were intravenously injected four times per group at low concentration (2.5×10⁶ cells), medium concentration (5×10⁶ cells), and high concentration (1×10⁷ cells) of CAR-positive cells per mouse (a of FIG. 33).

In addition, 4, 11, and 18 days after injection of U2932-Luc cell line into mice, CD19xCD22-CAR-NK cells were intravenously injected three times per group at a medium concentration (5×10⁶ cells) per CAR-positive cell (a of FIG. 34).

As a result, as shown in b of FIG. 32, b of FIG. 33, and b of FIG. 34, it was confirmed that tumor cells were killed by UCI-101 administration.

### Example 21

### Confirmation of Antigen-Specific Cytotoxicity of CD19xCD22-CAR-NK Cells (UCI-101)

### 21-1: Production of CD19−/−, CD22−/−, CD19−/−CD22−/−KO U2932 Cell Lines

The target cells, U2932 cells, express both CD19 and CD22. To confirm the antigen-specific cytotoxic effect of UCI-101, CD19−/−, CD22−/−, and CD19−/−CD22−/−KO U2932 cell lines were constructed. KO cell lines were constructed by electroporation using sgRNA (Alt-R®CRISPR-Cas9 sgRNA (IDT)) and Cas9 genes (Atl-R®Sp Cas9 Nuclease V3 (IDT) Cat #1081059) at 1450 V, 10 ms, 3 pulses.

The sequence information of the sgRNA used in the experiment was as follows:

(SEQ ID NO: 56)
CD19: 5′-CTGTGCTGCAGTGCCTCAA-3′
(SEQ ID NO: 57)
CD22: 5′-TCCTAGAGGGGGTTCCAATG-3′

Afterwards, KO cell lines were selected through the Positive selection or Negative selection method (MACS) using anti-CD19 microbeads (Miltenyi, #130-050-301) and anti-CD22 microbeads (Miltenyi, #130-046-401).

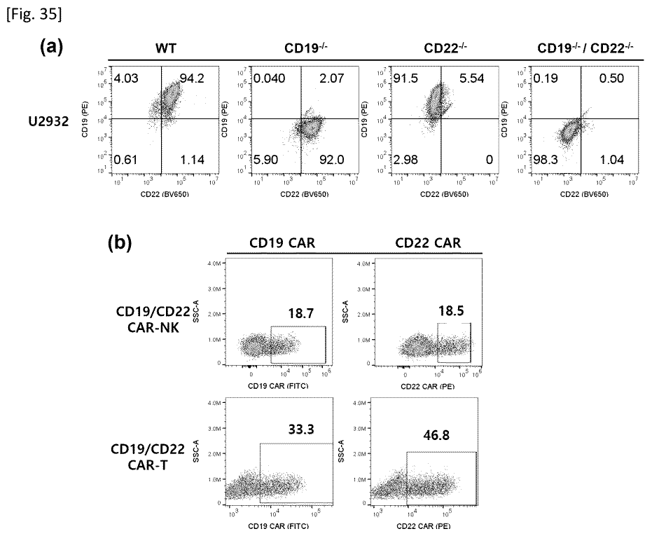

The selected cell lines were treated with Anti-CD19 (PE) and Anti-CD22 (BV650) antibodies, and the expression levels of CD19 and CD22 were confirmed using a flow cytometer (FIG. 35).

### 21-2: Production of CD19xCD22-CAR-T Cell

In addition, as a comparative example, CD19xCD22-CAR-T cells were prepared by the following method.

PBMCs were separated from blood aliquots obtained from leukapheresis of healthy donors by Ficoll-paque density gradient centrifugation, and then cryopreserved. The cryopreserved PBMCs were thawed, and T cells were isolated using the negative selection method (stem cell non-T cell Target kit). The isolated T cells were cultured for 2 days under 200 U/mL IL-2 conditions. On day 2, the cells were harvested, suspended to a concentration of 1×10⁷ cells/mL, and treated with CD19xCD22-CAR lentiviral vector at an MOI of 0.5 for 10 minutes. After 10 minutes, the medium was replaced with culture medium containing 200 U/ml IL-2, and on days 3 and 5, culture medium was added to a cell concentration of 5×10⁵ cells/mL. After additional culture up to day 7, the phenotypes of CD19xCD22-CAR-NK cells and CD19xCD22-CAR-T cells were analyzed by flow cytometry. As shown in b of FIG. 35, the % of CAR-positive cells in the CD56-positive, CD3-negative gate and the % of CAR-positive cells in the CD3-positive gate were represented.

### 21-3: Confirmation of Antigen-Specific Cytotoxicity

The target cells, WT-U2932 cell line and CD19, CD22 antigen KO U2932 cell line, were CTV labeled (100 nM) at a concentration of 1×10⁷ cells/ml in a 37° C. incubator for 20 minutes. After washing twice or more, they were co-cultured with CD19xCD22-CAR-NK cells and CD19xCD22-CAR-T cells for 4 hours. The cells were cultured in 24-well plates at a ratio of 5×10⁵ cells, calculated based on the total number of cells, so that the ratio of effector cells (CAR-NK cells and CAR-T cells) to target cells (E:T ratio) was 9:1, 3:1, and 11. After 4 hours, the cells were harvested, stained with PI, and analyzed by flow cytometry. The degree of PI expression was analyzed within the CTV-positive gate to measure the degree of cytotoxicity of the target cells.

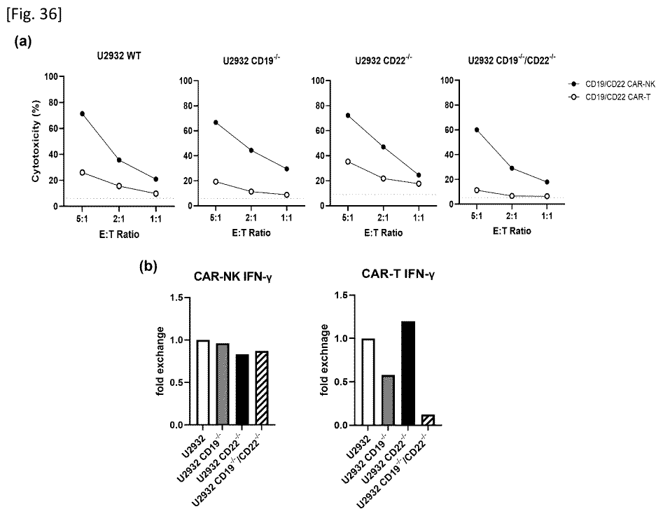

As a result, as shown in a of FIG. 36, CD19xCD22-CAR-NK cells were shown to have superior cytotoxicity against target cells compared to CD19xCD22-CAR-T cells, and in particular, were found to have high cytotoxicity against the WT-U2932 cell line expressing both CD19 and CD22 antigens.

### 21-4: Confirmation of Cytokine Secretion by CAR-NK cells and CAR-T Cells

The amount of IFN-gamma cytokine secreted from the supernatant obtained during cell recovery was measured through ELISA (Enzyme Linked Immunosorbent assay). ELISA was performed according to standard procedures (R&D systems), and the final absorbance was analyzed through a Multiplate Reader (Molecular Devices, spectramax id3 system). The amount of IFN-gamma was measured for the supernatant obtained after co-culture at E:T=5:1, which was used in the cytotoxicity confirmation test.

As a result, as shown in b of FIG. 36, it was confirmed that the cytokine secretion ability of CD19xCD22-CAR-NK cells was superior to that of CD19xCD22-CAR-T cells.

1. A method for producing CAR-NK cells targeting CD19 and CD22 comprising:
a step of treating peripheral blood mononuclear cells (PBMCs) expressing 70% or less of CD16, less than 10% of natural killer group 2D (NKG2D), 30% or less of CD57, 0.1% or more of low-density lipoprotein receptor (LDLR) and less than 10% of natural cytotoxicity triggering receptor 3 (NKp30) with dexamethasone to differentiate them into NK cells; and
a step of transducing the NK cells with a vector containing a polynucleotide encoding a bispecific chimeric antigen receptor (CAR) targeting CD19 and CD22;
wherein the bispecific chimeric antigen receptor (CAR) comprises a CD19-binding domain and a CD22-binding domain; a transmembrane domain; a costimulatory domain; and an intracellular signal transduction domain.

2. The method of claim 1, wherein the dexamethasone is treated at the beginning (day 0) of induction of NK cell differentiation, and the induction of differentiation is performed for 5 to 10 days in a medium containing dexamethasone.

3. The method of claim 1, wherein the NK cell is co-cultured with a feeder cell expressing one or more proteins selected from the group consisting of IL-2, IL-15, IL-21, OX40L (CD134 ligand), and lunasin for induction of NK cell differentiation.

4. The method of claim 1, wherein the vector is a viral vector, and the NK cells are treated with the viral vector at multiplicity of infection (MOI) of 3 to 7.

5. The method of claim 1, wherein the method further comprises prostaglandin E2 (PGE2) and polyoxyethylene-polyoxypropylene block copolymer to increase the expression rate of the chimeric antigen receptor (CAR) during the step of transducing.

6. The method of claim 1, wherein the CD19-binding domain and CD22-binding domain are linked in an order of:
a light chain variable region of an antibody specifically binding to CD19—a heavy chain variable region of an antibody specifically binding to CD22—a light chain variable region of an antibody specifically binding to CD22—a heavy chain variable region of an antibody specifically binding to CD19.

7. The method of claim 6, wherein the heavy chain variable region of the antibody specifically binding to CD22 comprises a CDR1 region represented by an amino acid of SEQ ID: 1, a CDR2 region represented by an amino acid of SEQ ID NO: 2 and a CDR3 region represented by an amino acid of SEQ ID NO: 3;
the light chain variable region of the antibody specifically binding to CD22 comprises a CDR1 region represented by an amino acid of SEQ ID NO: 4, a CDR2 region represented by an amino acid of SEQ ID NO: 5 and a CDR3 region represented by an amino acid of SEQ ID NO: 6;
the heavy chain variable region of the antibody specifically binding to CD19 comprises a CDR1 region represented by an amino acid of SEQ ID: 41, a CDR2 region represented by an amino acid of SEQ ID NO: 42 and a CDR3 region represented by an amino acid of SEQ ID NO: 43; and
the light chain variable region of the antibody specifically binding to CD19 comprises a CDR1 region represented by an amino acid of SEQ ID NO: 44, a CDR2 region represented by an amino acid of SEQ ID NO: 45 and a CDR3 region represented by an amino acid of SEQ ID NO: 46.

8. The method of claim 1, wherein the transmembrane domain is a protein selected from the group consisting of CD8α, CD4, CD28, CD137, CD80, CD86, CD152 and PD1,
the costimulatory domain is a protein selected from the group consisting of CD28, 4-1BB, OX-40 and ICOS, and
the intracellular signal transduction domain is CD3ζ.

9. The method of claim 1, wherein the bispecific chimeric antigen receptor (CAR) further comprises a hinge region between a C-terminus of the binding domain and an N-terminus of the transmembrane domain.

10. A CAR-NK cell targeting CD19 and CD22, produced by the method of claim 1.

11. The CAR-NK cell of claim 10, wherein the CAR-NK cell is characterized by low expression of CD16, NKG2D, LDLR, and NKp30, and high expression of CD57.

12. A CAR-NK cell targeting CD19 and CD22, comprising: a CD19-binding domain and a CD22-binding domain;
a transmembrane domain;
a costimulatory domain; and
an intracellular signal transduction domain,
wherein the CD22-binding domain is an antibody specifically binding to CD22 or a fragment thereof, comprising a heavy chain variable region comprising a CDR1 region represented by an amino acid of SEQ ID: 1, a CDR2 region represented by an amino acid of SEQ ID NO: 2 and a CDR3 region represented by an amino acid of SEQ ID NO: 3 and a light chain variable region comprising a CDR1 region represented by an amino acid of SEQ ID NO: 4, a CDR2 region represented by an amino acid of SEQ ID NO: 5 and a CDR3 region represented by an amino acid of SEQ ID NO: 6.

13. A CAR-NK cell targeting CD19 and CD22, comprising: a CD19-binding domain and a CD22-binding domain;
a transmembrane domain;
a costimulatory domain; and
an intracellular signal transduction domain,
wherein the CD22-binding domain is an antibody specifically binding to CD22 or a fragment thereof; and the heavy chain variable region of antibody specifically binding to CD22 comprises a CDR1 region represented by an amino acid of SEQ ID: 1, a CDR2 region represented by an amino acid of SEQ ID NO: 2 and a CDR3 region represented by an amino acid of SEQ ID NO: 3; and the light chain variable region of antibody specifically binding to CD22 comprises a CDR1 region represented by an amino acid of SEQ ID NO: 4, a CDR2 region represented by an amino acid of SEQ ID NO: 5 and a CDR3 region represented by an amino acid of SEQ ID NO: 6; and
wherein the CD19-binding domain is an antibody specifically binding to CD19 or a fragment thereof; and the heavy chain variable region of antibody specifically binding to CD19 comprises a CDR1 region represented by an amino acid of SEQ ID: 41, a CDR2 region represented by an amino acid of SEQ ID NO: 42 and a CDR3 region represented by an amino acid of SEQ ID NO: 43; and the light chain variable region of antibody specifically binding to CD19 comprises a CDR1 region represented by an amino acid of SEQ ID NO: 44, a CDR2 region represented by an amino acid of SEQ ID NO: 45 and a CDR3 region represented by an amino acid of SEQ ID NO: 46.

14. The CAR-NK cell targeting CD19 and CD22 of claim 12, wherein the CD19-binding domain and CD22-binding domain are linked in an order of:
a light chain variable region of an antibody specifically binding to CD19—a heavy chain variable region of an antibody specifically binding to CD22—a light chain variable region of an antibody specifically binding to CD22—a heavy chain variable region of an antibody specifically binding to CD19.

15. The CAR-NK cell targeting CD19 and CD22 of claim 13, wherein the CD19-binding domain and CD22-binding domain are linked in an order of:
a light chain variable region of an antibody specifically binding to CD19—a heavy chain variable region of an antibody specifically binding to CD22—a light chain variable region of an antibody specifically binding to CD22—a heavy chain variable region of an antibody specifically binding to CD19.

16. A pharmaceutical composition comprising the CAR-NK cell of claim 12 and a pharmaceutically acceptable carrier.

17. A pharmaceutical composition comprising the CAR-NK cell of claim 13 and a pharmaceutically acceptable carrier.

18. A method of treating a disease mediated by B cells, comprising administering or injecting the CAR-NK cell of claim 12 to a subject in need thereof.

19. A method of treating a disease mediated by B cells, comprising administering or injecting the CAR-NK cell of claim 13 to a subject in need thereof.

20. The method of claim 18, wherein the disease mediated by B cells is selected from the group consisting of tumors, lymphoma, non-Hodgkin' s lymphoma (NHL), aggressive NHL, relapsed aggressive NHL, relapsed indolent NHL, refractory NHL, refractory indolent NHL, chronic lymphocytic leukemia (CLL), small lymphocytic lymphoma (SLL), leukemia, hairy cell leukemia (HCL), acute lymphocytic leukemia (ALL), Burkitt's lymphoma, and mantle cell lymphoma.


SEQUENCE LISTING (57 sequences)

SEQ ID NO: 1 - protein, 9 aa, synthetic construct
  REGION 1..9: note = 2G1_VH_CDR1
GFSLTSYDI

SEQ ID NO: 2 - protein, 7 aa, synthetic construct
  REGION 1..7: note = 2G1_VH_CDR2
IWTGGGT

SEQ ID NO: 3 - protein, 12 aa, synthetic construct
  REGION 1..12: note = 2G1_VH_CDR3
VPHYYGYAMDYW

SEQ ID NO: 4 - protein, 6 aa, synthetic construct
  REGION 1..6: note = 2G1_VL_CDR1
QDINKY

SEQ ID NO: 5 - skipped

SEQ ID NO: 6 - protein, 8 aa, synthetic construct
  REGION 1..8: note = 2G1_VL_CDR3
LQYDNLLT

SEQ ID NO: 7 - protein, 117 aa, synthetic construct
  REGION 1..117: note = 2G1_VH
EVQLQESGPGLVAPSQSLSITCTVSGFSLTSYDISWIRQPPGKGLEWLGVIWTGGGTNYNSAFMSRLSISKDNSKSQVFLKMNSLQTDDTAIYYCVPHYYGYAMDYWGQGTSVTVSS

SEQ ID NO: 8 - protein, 106 aa, synthetic construct
  REGION 1..106: note = 2G1_VL
DIVLTQSPSSLSASLGGKVTITCKASQDINKYIAWYQHKPGKGPRLLIHYTSTLQPGIPSRFSGSGSGRDYSFSISNLEPEDIATYYCLQYDNLLTFGAGTKLELK

SEQ ID NO: 9 - DNA, 351 bp, synthetic construct
  misc_feature 1..351: note = 2G1_VH
  source 1..351: mol_type = other DNA
gaggtgcagctgcaggagtcaggacctggcctggtggcgccctcacagagcctgtccattacctgcactgtctctgggttctcattaaccagctatgatataagctggattcgccagccaccaggaaagggtctggagtggcttggagtaatatggactggtggaggcacaaattataattcagctttcatgtccagactgagcatcagcaaggacaactccaagagccaagttttcttaaaaatgaacagtctgcaaactgatgacacagccatatattactgtgtccctcattactacggctatgctatggactactggggtcaaggaacctcagtcaccgtctcctca

SEQ ID NO: 10 - DNA, 318 bp, synthetic construct
  misc_feature 1..318: note = 2G1_VL
  source 1..318: mol_type = other DNA
gatattgtgctgacccagtctccatcctcactgtctgcatctctgggaggcaaagtcaccatcacttgcaaggcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatctgctcacgttcggtgctgggaccaagctggagctgaaa

SEQ ID NO: 11 - protein, 117 aa, synthetic construct
  REGION 1..117: note = 2G1-V4_VH
EVQLQESGPGLVKPSQTLSLTCTVSGFSLTSYDISWIRQPPGKGLEWLGVIWTGGGTNYNSALKSRVTISKDNSKSQVSLKLSSVTAADTAVYYCVPHYYGYAMDYWGQGTTVTVSS

SEQ ID NO: 12 - protein, 106 aa, synthetic construct
  REGION 1..106: note = 2G1-V4_VL
EIVLTQSPATLSLSPGERATLSCRASQDINKYIAWYQQKPGQAPRLLIHYTSTRQTGIPARFSGSGSGRDYTLTISSLEPEDFAVYYCLQYDNLLTFGGGTKLEIK

SEQ ID NO: 13 - DNA, 351 bp, synthetic construct
  misc_feature 1..351: note = 2G1-V4_VH
  source 1..351: mol_type = other DNA
gaggtgcagctgcaggagagcggccccggcctggtgaagccgagccagactctttctctgacctgcaccgtgtccggcttctctcttacgagctacgacatctcgtggatccggcagccgcctgggaaaggcttagagtggctaggggtgatttggaccggcgggggtaccaactacaactccgcgctcaaatcccgcgtcactatttctaaggacaattccaagagccaggtctcgctgaagctctcgtccgtgaccgccgcggacaccgcagtttattactgcgtgcctcattactacggctacgccatggattattggggccagggcaccacagtaacagtcagctcc

SEQ ID NO: 14 - DNA, 318 bp, synthetic construct
  misc_feature 1..318: note = 2G1-V4_VL
  source 1..318: mol_type = other DNA
gagatcgtgctgactcagagcccggccacccttagcctgagtccaggcgagcgcgctacgttgtcatgccgagcttcccaggacattaacaagtacatcgcgtggtaccagcagaagcccggacaggccccccgcctgctcatccactacacctccacccgccagactggcatccctgccaggttttcaggctccggttctggccgtgactacaccctgaccatctctagtttggagcccgaagatttcgccgtgtactactgtctgcaatatgacaacctgctgaccttcggagggggtaccaagctggagatcaag

SEQ ID NO: 15 - protein, 117 aa, synthetic construct
  REGION 1..117: note = 2G1-V12_VH
EVQLKESGPVLVKPTETLTLTCTVSGFSLTSYDISWIRQPPGKALEWLGVIWTGGGTNYNSALKSRLTISKDNSKSQVVLTMTNMDPVDTATYYCVPHYYGYAMDYWGQGTTVTVSS

SEQ ID NO: 16 - protein, 106 aa, synthetic construct; identical to SEQ ID NO: 12
  REGION 1..106: note = 2G1-V12_VL

SEQ ID NO: 17 - DNA, 351 bp, synthetic construct
  misc_feature 1..351: note = 2G1-V12_VH
  source 1..351: mol_type = other DNA
gaggtgcagctgaaggagagcgggccggtgctggtgaagcctaccgagactctgaccctgacctgcactgtttccggcttctctctgacgagctacgacatcagttggatccgccagccacccggcaaagcgttggaatggctcggggtaatttggaccggtggcgggaccaactacaacagcgcgctcaaatcgcggctaaccatctcaaaggacaactccaagtcccaagtggtgttaactatgacaaatatggatccggtcgacaccgctacctattactgcgtgcctcattactacggctacgccatggattattggggccagggcacgaccgtgaccgtctccagt

SEQ ID NO: 18 - DNA, 318 bp, synthetic construct
  misc_feature 1..318: note = 2G1-V12_VL
  source 1..318: mol_type = other DNA
gagatcgtgttgacccagagccctgccacgctgagcctgtcccccggggagcgcgccactctttcgtgtagggcttcccaggacattaacaagtacatcgcatggtaccagcagaagcccggacaggccccccgcctgctcatccactacacatccacccgccagacaggcatcccggctcgattctctggttctggcagcggtcgtgattacacccttactatttcttccctggagccagaggactttgcggtgtactactgcctgcagtatgacaacctgctgaccttcggcggaggcaccaagctggagatcaag

SEQ ID NO: 19 - protein, 15 aa, synthetic construct
  REGION 1..15: note = scfv linker
GGGGSGGGGSGGGGS

SEQ ID NO: 20 - DNA, 45 bp, synthetic construct
  misc_feature 1..45: note = scfv linker
  source 1..45: mol_type = other DNA
ggcggaggcggcagcggcggaggcggctctggcggcggcgggagc

SEQ ID NO: 21 - DNA, 45 bp, synthetic construct
  misc_feature 1..45: note = scfv linker
  source 1..45: mol_type = other DNA
gggggcggcggatctggtggtggaggctccggcggtggcggttcc

SEQ ID NO: 22 - DNA, 45 bp, synthetic construct
  misc_feature 1..45: note = scfv linker
  source 1..45: mol_type = other DNA
ggcggcggtggctccggtggaggcggatctggcggtggcgggtcg

SEQ ID NO: 23 - protein, 238 aa, synthetic construct
  REGION 1..238: note = 2G1 scfv
DIVLTQSPSSLSASLGGKVTITCKASQDINKYIAWYQHKPGKGPRLLIHYTSTLQPGIPSRFSGSGSGRDYSFSISNLEPEDIATYYCLQYDNLLTFGAGTKLELKGGGGSGGGGSGGGGSEVQLQESGPGLVAPSQSLSITCTVSGFSLTSYDISWIRQPPGKGLEWLGVIWTGGGTNYNSAFMSRLSISKDNSKSQVFLKMNSLQTDDTAIYYCVPHYYGYAMDYWGQGTSVTVSS

SEQ ID NO: 24 - DNA, 714 bp, synthetic construct
  misc_feature 1..714: note = 2G1 scfv
  source 1..714: mol_type = other DNA
gatatcgtgctgacccagagcccttctagcctgagcgcctctctgggcggcaaggtgaccatcacctgcaaggccagccaggacatcaacaagtacatcgcctggtaccagcacaagcctggaaaaggccctagactgctgatccactacaccagcacactccagcctggcatccccagcagattcagcggatctggaagcggcagagattacagcttcagcatttctaatctggaacctgaggacatcgctacatactattgtctgcagtacgacaacctgctgaccttcggcgctggcaccaagctggagctgaaaggcggaggcggcagcggcggaggcggctctggcggcggcgggagcgaggtgcagctgcaggagagcggccccggactggtcgccccaagccaatctctgtccatcacatgcaccgtgtccggctttagcctgacatcatatgatatcagctggatccggcagccacctggcaagggcctggaatggctgggcgttatctggaccggcggaggcacaaactacaactccgccttcatgagccggctgagcatctctaaggacaatagcaagtcccaggtgttcctgaagatgaacagcctgcaaacagacgacaccgccatctactactgcgtgccccactactacggctacgccatggactactggggccagggcaccagcgtgaccgtgtccagc

SEQ ID NO: 25 - protein, 238 aa, synthetic construct
  REGION 1..238: note = 2G1-V4 scfv
EIVLTQSPATLSLSPGERATLSCRASQDINKYIAWYQQKPGQAPRLLIHYTSTRQTGIPARFSGSGSGRDYTLTISSLEPEDFAVYYCLQYDNLLTFGGGTKLEIKGGGGSGGGGSGGGGSEVQLQESGPGLVKPSQTLSLTCTVSGFSLTSYDISWIRQPPGKGLEWLGVIWTGGGTNYNSALKSRVTISKDNSKSQVSLKLSSVTAADTAVYYCVPHYYGYAMDYWGQGTTVTVSS

SEQ ID NO: 26 - DNA, 714 bp, synthetic construct
  misc_feature 1..714: note = 2G1-V4 scfv
  source 1..714: mol_type = other DNA
gagatcgtgctgactcagagcccggccacccttagcctgagtccaggcgagcgcgctacgttgtcatgccgagcttcccaggacattaacaagtacatcgcgtggtaccagcagaagcccggacaggccccccgcctgctcatccactacacctccacccgccagactggcatccctgccaggttttcaggctccggttctggccgtgactacaccctgaccatctctagtttggagcccgaagatttcgccgtgtactactgtctgcaatatgacaacctgctgaccttcggagggggtaccaagctggagatcaaggggggcggcggatctggtggtggaggctccggcggtggcggttccgaggtgcagctgcaggagagcggccccggcctggtgaagccgagccagactctttctctgacctgcaccgtgtccggcttctctcttacgagctacgacatctcgtggatccggcagccgcctgggaaaggcttagagtggctaggggtgatttggaccggcgggggtaccaactacaactccgcgctcaaatcccgcgtcactatttctaaggacaattccaagagccaggtctcgctgaagctctcgtccgtgaccgccgcggacaccgcagtttattactgcgtgcctcattactacggctacgccatggattattggggccagggcaccacagtaacagtcagctcc

SEQ ID NO: 27 - protein, 238 aa, synthetic construct
  REGION 1..238: note = 2G1-V12 scfv
EIVLTQSPATLSLSPGERATLSCRASQDINKYIAWYQQKPGQAPRLLIHYTSTRQTGIPARFSGSGSGRDYTLTISSLEPEDFAVYYCLQYDNLLTFGGGTKLEIKGGGGSGGGGSGGGGSEVQLKESGPVLVKPTETLTLTCTVSGFSLTSYDISWIRQPPGKALEWLGVIWTGGGTNYNSALKSRLTISKDNSKSQVVLTMTNMDPVDTATYYCVPHYYGYAMDYWGQGTTVTVSS

SEQ ID NO: 28 - DNA, 714 bp, synthetic construct
  misc_feature 1..714: note = 2G1-V12 scfv
  source 1..714: mol_type = other DNA
gagatcgtgttgacccagagccctgccacgctgagcctgtcccccggggagcgcgccactctttcgtgtagggcttcccaggacattaacaagtacatcgcatggtaccagcagaagcccggacaggccccccgcctgctcatccactacacatccacccgccagacaggcatcccggctcgattctctggttctggcagcggtcgtgattacacccttactatttcttccctggagccagaggactttgcggtgtactactgcctgcagtatgacaacctgctgaccttcggcggaggcaccaagctggagatcaagggcggcggtggctccggtggaggcggatctggcggtggcgggtcggaggtgcagctgaaggagagcgggccggtgctggtgaagcctaccgagactctgaccctgacctgcactgtttccggcttctctctgacgagctacgacatcagttggatccgccagccacccggcaaagcgttggaatggctcggggtaatttggaccggtggcgggaccaactacaacagcgcgctcaaatcgcggctaaccatctcaaaggacaactccaagtcccaagtggtgttaactatgacaaatatggatccggtcgacaccgctacctattactgcgtgcctcattactacggctacgccatggattattggggccagggcacgaccgtgaccgtctccagt

SEQ ID NO: 29 - DNA, 1178 bp, synthetic construct
  misc_feature 1..1178: note = EF1 promoter
  source 1..1178: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 30 - DNA, 63 bp, synthetic construct
  misc_feature 1..63: note = signal peptide
  source 1..63: mol_type = other DNA
atggccttaccagtgaccgccttgctcctgccgctggccttgctgctccacgccgccaggccg

SEQ ID NO: 31 - DNA, 135 bp, synthetic construct
  misc_feature 1..135: note = CD8 Hinge
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 32 - DNA, 72 bp, synthetic construct
  misc_feature 1..72: note = transmembrane domain
  source 1..72: mol_type = other DNA
atctacatctgggcgcccttggccgggacttgtggggtccttctcctgtcactggttatcaccctttactgc

SEQ ID NO: 33 - DNA, 126 bp, synthetic construct
  misc_feature 1..126: note = 4-1BB
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 34 - DNA, 336 bp, synthetic construct
  misc_feature 1..336: note = CD3 zeta
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 35 - DNA, 591 bp, synthetic construct
  misc_feature 1..591: note = WPRE
  source 1..591: mol_type = other DNA
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 36 - protein, 21 aa, synthetic construct
  REGION 1..21: note = signal peptide
MALPVTALLLPLALLLHAARP

SEQ ID NO: 37 - protein, 45 aa, synthetic construct
  REGION 1..45: note = CD8 Hinge
TTTPAPRPPTPAPTIASQPLSLRPEACRPAAGGAVHTRGLDFACD

SEQ ID NO: 38 - protein, 24 aa, synthetic construct
  REGION 1..24: note = transmembrane domain
IYIWAPLAGTCGVLLLSLVITLYC

SEQ ID NO: 39 - protein, 42 aa, synthetic construct
  REGION 1..42: note = 4-1BB
KRGRKKLLYIFKQPFMRPVQTTQEEDGCSCRFPEEEEGGCEL

SEQ ID NO: 40 - protein, 112 aa, synthetic construct
  REGION 1..112: note = CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 8 aa, synthetic construct
  REGION 1..8: note = CD19_VH_CDR1
GVSLPDYG

SEQ ID NO: 42 - protein, 7 aa, synthetic construct
  REGION 1..7: note = CD19_VH_CDR2
IWGSETT

SEQ ID NO: 43 - protein, 14 aa, synthetic construct
  REGION 1..14: note = CD19_VH_CDR3
AKHYYYGGSYAMDY

SEQ ID NO: 44 - protein, 6 aa, synthetic construct
  REGION 1..6: note = CD19_VL_CDR1
QDISKY

SEQ ID NO: 45 - skipped

SEQ ID NO: 46 - protein, 9 aa, synthetic construct
  REGION 1..9: note = CD19_VL_CDR3
QQGNTLPYT

SEQ ID NO: 47 - protein, 119 aa, synthetic construct
  REGION 1..119: note = CD19_VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 48 - protein, 128 aa, synthetic construct
  REGION 1..128: note = CD19_VL
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 49 - DNA, 360 bp, synthetic construct
  misc_feature 1..360: note = CD19_VH
  source 1..360: mol_type = other DNA
tctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcc

SEQ ID NO: 50 - DNA, 384 bp, synthetic construct
  misc_feature 1..384: note = CD19_VL
  source 1..384: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcaca

SEQ ID NO: 51 - protein, 5 aa, synthetic construct
  REGION 1..5: note = Bi-specific CART linker
GGGGS

SEQ ID NO: 52 - DNA, 15 bp, synthetic construct
  misc_feature 1..15: note = Bi-specific CART linker
  source 1..15: mol_type = other DNA
ggcgggggaggctct

SEQ ID NO: 53 - DNA, 15 bp, synthetic construct
  misc_feature 1..15: note = Bi-specific CART linker
  source 1..15: mol_type = other DNA
ggcggagggggctcc

SEQ ID NO: 54 - protein, 18 aa, synthetic construct
  REGION 1..18: note = Bi-specific CART linker
GSTSGSGKPGSGEGSTKG

SEQ ID NO: 55 - DNA, 54 bp, synthetic construct
  misc_feature 1..54: note = Bi-specific CART linker
  source 1..54: mol_type = other DNA
ggctccaccagtggttctgggaaacctggtagtggggagggctcgacgaagggg

SEQ ID NO: 56 - RNA, 19 nt, synthetic construct
  source 1..19: mol_type = other RNA; note = CD19 sgRNA
ctgtgctgcagtgcctcaa

SEQ ID NO: 57 - RNA, 20 nt, synthetic construct
  source 1..20: mol_type = other RNA; note = CD22 sgRNA
tcctagagggggttccaatg